(19) United States
(12) Patent Application Publication
(10) Pub. No.: US 2016/0296441 A1
(43) Pub. Date: Oct. 13, 2016

(54) CONDITIONER FOR KERATIN FIBERS HAVING A SILICONE SUBSTITUTE

(71) Applicant: Henkel AG & Co. KGaA, Duesseldorf (DE)

(72) Inventors: Edith von Aspern, Hanstedt (DE); Thomas Hippe, Appen (DE)

(73) Assignee: Henkel AG & Co. KGaA, Duesseldorf (DE)

(21) Appl. No.: 15/185,237

(22) Filed: Jun. 17, 2016

Related U.S. Application Data
(63) Continuation of application No. PCT/EP2014/076859, filed on Dec. 8, 2014.

(30) Foreign Application Priority Data
Dec. 20, 2013 (DE) 10 2013 226 944.7

(51) Int. Cl. A61K 8/41; A61K 8/37; A61K 8/46; A61Q 5/00; A61Q 5/12

(52) U.S. Cl. CPC A61K 8/416; A61Q 5/004; A61Q 5/12; A61K 8/463; A61K 8/37; A61K 2800/5922; A61K 2800/596

(57) ABSTRACT

Compositions for treating keratin fibers, in particular human hair, significantly improve the conditioning properties and the shine and feel of keratin fibers. The compositions include, as essential ingredients, at least one selected cationic surfactant and at least one selected ester of 3-methyl-1-butanol.

### FIELD OF THE INVENTION

The present invention generally relates to compositions for treating keratin fibers, in particular human hair, which greatly improve the conditioning properties and the luster and feel of such fibers.

### BACKGROUND OF THE INVENTION

The cosmetic treatment of keratin fibers, in particular human hair, is an important part of human bodily care. Thus, human hair nowadays is treated in various ways with cosmetic hair preparations.

There is also a need for active substances or active substance combinations for cosmetic agents having good care properties and good biodegradability. In addition, compatibility of the cosmetic compositions is an extremely important criterion.

Compositions for use on keratin fibers, in particular human hair, must not only have good cleaning, conditioning, and care capabilities, but also should be well tolerated, and should not result in excessive oil removal, dryness, or split ends of the hair, even with frequent use. The feel of the keratin fibers after cosmetic treatments is an important criterion for whether the composition in question is perceived as pleasant by the consumer. The sensory properties and in particular the feel of a composition are thus important effects which the consumer can experience. For this reason, compositions which not only provide care for the keratin fibers, in particular human hair, but also noticeably and perceptibly change the surface of keratin fibers, in particular human hair, due to their sensory and in particular haptic properties, are sought in particular. Furthermore, the compositions should regenerate and balance the structure, in particular inside the keratin fibers, in particular the human hair. At the same time, the compositions should be easily and cost-effectively producible. Silicones are known for their excellent hair conditioning properties. However, silicones have the drawback of requiring a high level of energy consumption for their synthesis, and sometimes being very persistent in the environment. Therefore, there is a need for silicone-free cosmetic agents. For this reason, there is a further need to provide a good hair conditioning agent which offers superior conditioning properties without using silicones.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic composition for conditioning keratin fibers includes in a cosmetic carrier at least one cationic surfactant of formula (Tkat1-3),

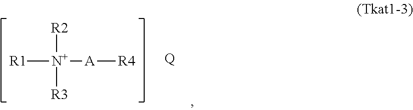

where R1, R2, and R3 each independently mean: hydrogen, with the condition that R4 stands for R8-NH—, or a free electron pair, with the condition that R4 stands for R8-NH—, and only one of the moieties R1, R2, R3 stands for a free electron pair and Q is not required, or a branched or linear alkyl moiety, including 1 to 4 carbon atoms, which may include at least one hydroxyl group, or a saturated or unsaturated, branched or linear alkyl moiety including 6 to 30 carbon atoms, which may include at least one hydroxyl group, or the moiety (-A-R4), with the condition that no more than two of the moieties R1, R2, or R3 may stand for this moiety, or R2 and R3 form a cyclic or heterocyclic, optionally unsaturated alkylene group which may be substituted with a saturated or unsaturated, branched or linear alkyl moiety including 6 to 20 carbon atoms, for example an imidazolium group, and A stands for: —(CH₂)ₙ—, where n=1 to 20, preferably n=1 to 10, and particularly preferably n=2-3, or —(CH₂—CHR₅—O)ₙ, where n=1 to 200, preferably 1 to 100, particularly preferably 1 to 50, and particularly preferably 1 to 20, where R5 means hydrogen, methyl, or ethyl, or a hydroxyalkylene group including one to four, preferably two to three, carbon atoms, which may be branched or linear and which include one to three hydroxy groups, preferably a group selected from >CHOH, —CH₂CHOH—, —CHOHCHOH—, —CH₂CHOHCH₂—, >C(CH₂OH)₂, >C(CH₂OH)₂, —CH₂CHOHCHOH—, —CH₂CH(CH₂OH)—, and hydroxybutyl moieties, and R4 stands for: R6-COO—, where R6 represents a saturated or unsaturated, branched or linear alkyl moiety or a cyclic, saturated or unsaturated alkyl moiety including 6 to 30, preferably 11 to 21, particularly preferably 15 to 17, carbon atoms, which includes at least one hydroxy group and optionally may optionally also be oxalkylated with 1 to 100 ethylene oxide units and/or 1 to 100 propylene oxide units, or R7-CO—, where R7 is a saturated or unsaturated, branched or linear alkyl moiety or a cyclic, saturated or unsaturated alkyl moiety including 6 to 30 carbon atoms, which may include at least one hydroxy group and which may optionally also be oxalkylated with 1 to 100 ethylene oxide units and/or 1 to 100 propylene oxide units, or R8-NH where R8 represents a linear or branched, acyl or alkyl moiety in each case including 6 to 30 C atoms, which may be saturated or unsaturated and/or substituted with at least one OH group, and Q stands for a physiologically compatible organic or inorganic anion, if none of the moieties R1, R2, R3 stands for a free electron pair; and in addition at least one ester of 3-methyl-1-butanol with an acid of the general formula R′COOH, where R′ represents a saturated or unsaturated, branched or linear alkyl moiety including 7 to 29, preferably 11 to 21, carbon atoms, and which may include at least one hydroxy group, wherein the at least one cationic surfactant of formula (Tkat1-3) is preferably included in a total quantity of 0.2 to 10.0% by weight, particularly preferably 1.0 to 5.0% by weight, extremely preferably 1.5 to 4.0% by weight, in each case based on the weight of the composition.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now very unexpectedly been found that a cosmetic composition which includes at least one selected cationic surfactant and at least one selected ester of 3-methyl-1-butanol in a cosmetic carrier ideally meets the stated objects.

Use of this active substance combination results in surprisingly good properties of the treated hair, in particular improved combability, luster, and elasticity, as well as greatly increased wash resistance of the color of dyed hair, in addition to a longer shelf life while at the same time providing better shaping capability for wave processes such as water wave and permanent wave, or for straightening agents for curly hair.

The subject matter of the present invention relates to a cosmetic composition for conditioning keratin fibers, which in a cosmetic carrier includes
a) at least one cationic surfactant of formula (Tkat1-3),

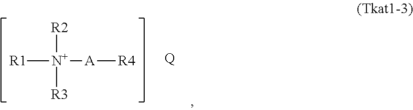

where R1, R2, and R3 each independently mean:
   hydrogen, with the condition that R4 stands for R8-NH—, or
   a free electron pair, with the condition that R4 stands for R8-NH—, and only one of the moieties R1, R2, R3 stands for a free electron pair and Q is not required, or
   a branched or linear alkyl moiety, including 1 to 4 carbon atoms, which may include at least one hydroxyl group, or
   a saturated or unsaturated, branched or linear alkyl moiety including 6 to 30 carbon atoms, which may include at least one hydroxyl group, or
   the moiety (-A-R4), with the condition that no more than two of the moieties R1, R2, or R3 may stand for this moiety, or
   R2 and R3 form a cyclic or heterocyclic, optionally unsaturated alkylene group which may be substituted with a saturated or unsaturated, branched or linear alkyl moiety including 6 to 20 carbon atoms, for example an imidazolium group,
and A stands for:
   1) —(CH₂)ₙ—, where n=1 to 20, preferably n=1 to 10, and particularly preferably n=2-3, or
   2) —(CH₂—CHR₅—O)ₙ, where n=1 to 200, preferably 1 to 100, particularly preferably 1 to 50, and particularly preferably 1 to 20, where R5 means hydrogen, methyl, or ethyl, or
   3) a hydroxyalkylene group including one to four, preferably two to three, carbon atoms, which may be branched or linear and which include one to three hydroxy groups, preferably a group selected from >CHOH, —CH₂CHOH—, —CHOHCHOH—, —CH₂CHOHCH₂—, >C(CH₂OH)₂, >C(CH₂OH)₂, —CH₂CHOHCHOH—, —CH₂CH(CH₂OH)—, and hydroxybutyl moieties,
and R4 stands for:
   R6-COO—, where R6 represents a saturated or unsaturated, branched or linear alkyl moiety or a cyclic, saturated or unsaturated alkyl moiety including 6 to 30, preferably 11 to 21, particularly preferably 15 to 17, carbon atoms, which includes at least one hydroxy group and optionally may also be oxalkylated with 1 to 100 ethylene oxide units and/or 1 to 100 propylene oxide units, or
   R7-CO—, where R7 is a saturated or unsaturated, branched or linear alkyl moiety or a cyclic, saturated or unsaturated alkyl moiety including 6 to 30 carbon atoms, which may include at least one hydroxy group and which may optionally also be oxalkylated with 1 to 100 ethylene oxide units and/or 1 to 100 propylene oxide units, or
   R8-NH—, where R8 represents a linear or branched, acyl or alkyl moiety in each case including 6 to 30 C atoms, which may be saturated or unsaturated and/or substituted with at least one OH group, and
Q stands for a physiologically compatible organic or inorganic anion, if none of the moieties R1, R2, R3 stands for a free electron pair,
and in addition
b) at least one ester of 3-methyl-1-butanol with an acid of the general formula R′COOH, where R′ represents a saturated or unsaturated, branched or linear alkyl moiety including 7 to 29, preferably 11 to 21, carbon atoms, and which may include at least one hydroxy group,
   wherein the at least one cationic surfactant of formula (Tkat1-3) is preferably included in a total quantity of 0.2 to 10.0% by weight, particularly preferably 1.0 to 5.0% by weight, extremely preferably 1.5 to 4.0% by weight, in each case based on the weight of the composition.

Within the meaning of the present invention, examples of hair treatment agents are hair shampoos, hair conditioners, conditioning shampoos, hair rinses, hair masks, hair packs, hair tonics, hair-coloring shampoos, or combinations thereof. In particular, hair conditioning compositions such as hair rinses, hair masks, hair packs, hair oils and lotions, and as leave-on products, i.e., products that remain on the hair until the next hair washing, and also as rinse-off products, i.e., products that are to be rinsed out a few seconds to several hours after application, are understood to mean hair treatment agents according to the invention.

According to the invention, “combability” is understood to mean the combability of wet fibers as well as the combability of dry fibers.

“Feel” is defined as the tactility of a collection of fibers; the parameters of volume and smoothness are felt and assessed by sensory means by those skilled in the art.

“Shaping” is understood to mean the capability of imparting a change in shape to a collection of previously treated keratin-including fibers, in particular human hair. In hair cosmetics, this is also referred to as stylability.

Within the meaning of the invention, “restructuring” is understood to mean a reduction in the damage to keratin fibers resulting from various influences. For example, restoration of the natural strength plays an important role here. Restructured fibers are characterized by enhanced luster, improved hold, and better combability. In addition, they have greater strength and elasticity. Moreover, successful restructuring may be physically demonstrated as an increase in the melting point in comparison to the damaged fiber. The higher the melting point of the hair, the stronger the structure of the fiber.

Within the meaning of the invention, wash fastness is the retention of the original coloring with regard to shading and/or intensity when the keratin fiber is exposed to the repeated influence of aqueous agents, in particular surfactant-including agents such as shampoos.

In addition, the compositions according to the invention including the active substance combination according to the invention are characterized by a greatly improved condition of the keratin fibers with regard to the moisture balance of the keratin fibers. Furthermore, the active substance combination according to the invention results in considerable protection of the keratin fibers from the effects of heat, for example during blow drying of keratin fibers. The protection of the surface of keratin fibers from the effects of heat is of major importance in particular when straightening irons or hair dryers are used. Lastly, it has surprisingly been found that the compositions according to the invention result in greatly retarded resoiling of the keratin fibers.

The cosmetic carrier according to the invention is preferably an aqueous cosmetic carrier including at least 30% by weight water, based on the weight of the composition according to the invention.

Within the meaning of the present invention, aqueous-alcoholic cosmetic carriers are understood to mean aqueous solutions including 3 to 50% by weight, based on the weight of the composition according to the invention, of at least one C₂-C₆ alcohol, in particular ethanol or propanol, isopropanol, 1,2-propylene glycol, butanol, isobutanol, tert-butanol, n-pentanol, isopentanols, in particular 3-methyl-1-butanol, n-hexanols, isohexanols, ethylene glycol, glycerin, 1,2-pentanediol, 1,5-pentanediol, 2-methyl-2,4-pentanediol, 1,2-hexanediol, or 1,6-hexanediol. The agents according to the invention may additionally include further organic solvents, for example methoxybutanol, benzyl alcohol, or ethyl diglycol. All water-soluble organic solvents are preferred.

Cationic surfactants of formula (Tkat 1-3) are the first group of mandatory ingredients of the compositions according to the invention.

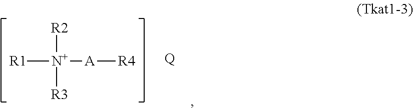

The meaning of the individual substituents has already been described above.

Q stands for a physiologically compatible anion, for example halides such as chloride or bromide, and for methosulfate.

Preferred as the physiologically compatible anion Q are halide ions, sulfate ions, phosphate ions, methosulfate ions, ethosulfate ions, and organic ions such as lactate, citrate, tartrate, and acetate ions. Methosulfates, ethosulfates, and halide ions, in particular chloride, are preferred.

The at least one cationic surfactant of formula (Tkat 1-3) is preferably included in the compositions according to the invention in a total quantity of 0.2 to 10.0% by weight, particularly preferably 1.0 to 5.0% by weight, very preferably 1.5 to 4.0% by weight, in each case based on the weight of the composition.

A first preferred embodiment of the invention is characterized in that the at least one cationic surfactant is selected from compounds of formula (Tkat1-3), wherein

R1 and R2 and R3 stand for methyl and
A stands for a linear hydroxyalkylene group which includes three carbon atoms and has a hydroxy group, and preferably stands for the group —CH₂CHOHCH₂—, and
R4 stands for R6-COO— and
R6 stands for a saturated, linear alkyl moiety including 11 to 21 carbon atoms and
Q stands for methosulfates, ethosulfates, or chloride.

A particularly preferred cationic surfactant of this type is selected from 3-behenoyloxy-2-hydroxypropyltrimethylammonium chloride.

Cationic surfactants of this type, preferably selected from 3-behenoyloxy-2-hydroxypropyltrimethylammonium chloride, are preferably included in the compositions according to the invention in a total quantity of 0.2 to 5.0% by weight, particularly preferably 0.5 to 3.0% by weight, extremely preferably 1.0 to 2.0% by weight, in each case based on the weight of the composition.

Another preferred embodiment of the invention is characterized in that the at least one cationic surfactant is selected from compounds of formula (Tkat1-3), wherein

R1 stands for methyl and
R2 stands for 2-hydroxyethyl and
R3 stands for the moiety (-A-R4) and
A stands for the group —CH₂—CH₂— and
R4 stands for R6-COO— and
R6 stands for a saturated, linear alkyl moiety including 11 to 21, preferably 15 to 17, carbon atoms and
Q stands for methosulfates, ethosulfates, or chloride.

A particularly preferred cationic surfactant of this type is selected from distearoylethyl hydroxyethylmonium methosulfate. Another, likewise particularly preferred cationic surfactant of this type is selected from dicocoylethyl hydroxyethylmonium methosulfate.

Cationic surfactants of this type, preferably selected from distearoylethyl hydroxyethylmonium methosulfate and dicocoylethyl hydroxyethylmonium methosulfate, are preferably included in the compositions according to the invention in a total quantity of 0.2 to 5.0% by weight, particularly preferably 0.5 to 3.0% by weight, extremely preferably 1.0 to 2.0% by weight, in each case based on the weight of the composition.

Such surfactants are also referred to as esterquats, and are marketed under the trade names Rewoquat®, Stepantex®, Dehyquart®, and Armocare®, for example. The products Quartamin BTC 131, Armocare® VGH-70, an N,N-bis(2-palmitoyloxyethyl)dimethylammonium chloride, and Dehyquart® F-75, Dehyquart® C-4046, Dehyquart® L80, Dehyquart® F-30, Dehyquart® AU-35, Rewoquat® WE18, Rewoquat® WE38 DPG, and Stepantex® VS 90 are examples of such esterquats.

Another preferred embodiment of the invention is characterized in that the at least one cationic surfactant is selected from compounds of formula (Tkat1-3), wherein

R1 stands for methyl and
R2 and R3 form a heterocyclic unsaturated alkylene group which is substituted with a saturated or unsaturated, linear alkyl moiety including 6 to 20 carbon atoms,
A stands for the group —CH₂—CH₂— and
R4 stands for R8-NH— and
R8 stands for a linear acyl moiety including 6 to 30 C atoms, preferably 10 to 20 C atoms, which is saturated or unsaturated, and
Q stands for methosulfates, ethosulfates, or chloride.

A particularly preferred cationic surfactant of this type is selected from Quaternium-87. Another, likewise particularly preferred cationic surfactant of this type is selected from Quaternium-27.

Cationic surfactants of this type, preferably selected from Quaternium-87 and Quaternium-27, are preferably included in the compositions according to the invention in a total quantity of 0.2 to 5.0% by weight, particularly preferably 0.5 to 3.0% by weight, extremely preferably 1.0 to 2.0% by weight, in each case based on the weight of the composition.

Another preferred embodiment of the invention is characterized in that the at least one cationic surfactant is selected from compounds of formula (Tkat1-3), wherein

R1 and R2 stand for methyl and
R3 stands for hydrogen or a free electron pair and
A stands for the group —CH₂—CH₂—CH₂— and
R4 stands for R8-NH— and
R8 stands for a linear or branched acyl moiety including 6 to 30 C atoms, preferably 10 to 20 C atoms, particularly preferably 12 to 18 C atoms, which is saturated or unsaturated, and
Q stands for methosulfates, ethosulfates, or chloride if R3 stands for hydrogen.

Cationic surfactants of this type are also referred to as amidoamines. In neutral or alkaline solution they are present in uncharged form; i.e., R3 stands for a free electron pair, and the presence of an anion Q is not required. When the pH is acidic, the amidoamines are present in quaternized form; i.e., R3 stands for hydrogen, and an anion Q is required for charge equalization.

A composition is preferred in which the amidoamine and/or the quaternized amidoamine according to formula (Tkat 1-3) is an amidoamine and/or a quaternized amidoamine, where R1 means a branched or linear, saturated or unsaturated acyl moiety including 6 to 30 C atoms, which may include at least one OH group. A fatty acid moiety composed of oils and waxes, in particular natural oils and waxes, is preferred. Examples include lanolin, beeswax, or candelilla wax.

Also preferred are amidoamines and/or quaternized amidoamines in which R1, R2, and/or R3 in formula (Tkat 1-3) mean a moiety according to the general formula CH₂CH₂OR5, where R5 means alkyl moieties including 1 to 4 carbon atoms, hydroxyethyl, or hydrogen. The preferred value of n in the placeholder A of general formula (Tkat 1-3) is an integer between 2 and 5.

The alkylamidoamines may be present as such, or may also be converted to a quaternary compound in the composition by protonation in an appropriately acidic solution. Cationic acylamidoamines are preferred according to the invention.

Preferred amidoamines according to the invention are lauramidopropyl dimethylamine (Mackine 801, for example), lauramidopropyl dimethylamine propionate, stearamidopropyl dimethylamine (Adogen® S18V, Tego® Amid S 18, or Incromine® SB, for example), myristamidopropyl dimethylamine (Schercodine M, for example), stearamidoethyl diethylamine (Lexamine 22, for example), stearamidoethyl diethylamine phosphate, cocamidopropyl dimethylamine (Mackine® 101, for example), ricinolamidopropyl dimethylamine (Mackine® 201, for example), isostearamidopropyl dimethylamine (Mackine 401, for example), oleamidopropyl dimethylamine (Mackine® 501, for example), behenamidopropyl dimethylamine (Mackine 601, Incromine® BD, for example), cocamidopropyl dimethylamine propionate (Mackalene® 117, for example), cocamidopropyl dimethylamine lactate (Mackalene® 116, for example), ricinoleamidopropyl dimethylamine lactate (Mackalene 216, for example), stearamidopropyl dimethylamine lactate (Mackalene 316), behenamidopropyl dimethylamine lactate (Mackalene® 616, for example), sunflower seed amidopropyl dimethylamine lactate (Mackalene 1216, for example), palmamidopropyl dimethylamine, palmamidopropyl dimethylamine lactate, palmamidopropyl dimethylamine propionate, oleamidopropyl dimethylamine glycolate, and oleamidopropyl dimethylamine lactate, and examples of permanent cationic amidoamines are: Quaternium-33 (Swanol® Lanoquat DES-50, for example), behenamidopropyl ethyldimonium ethosulfate (Schercoquat® BAS, for example), behenamidopropyl PG-dimonium chloride (Lexquat® AMG-BEO, for example), oleamidopropyl ethyldimonium ethosulfate, oleamidopropyl PG-dimonium chloride (Lexquat® AMG-O, for example), cocamidopropyl ethyldimonium ethosulfate (Schercoquat® CAS, for example), cocamidopropyltrimonium chloride (Empigen® CSC, for example), ricinoleamidopropylethyldimonium ethosulfate, rinoleamidopropyltrimonium chloride, ricinoleamidopropyltrimonium methosulfate (Rewoquat® RTM 50, for example), stearamidopropyl ethyldimonium ethosulfate (Schercoquat® SAS, for example), stearamidopropyl trimonium methosulfate (Catagene® SA-70, for example) or undecyleneamidopropyltrimonium methosulfate (Rewoquat® UTM 50, for example), lauramidopropyl PG-dimonium chloride, canolamidopropyl ethyldimonium ethosulfate (Schercoquat® COAS, for example).

Preferred amidoamines are lauramidopropyl dimethylamine, myristamidopropyl dimethylamine, stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine, palmamidopropyl dimethylamine, Quaternium-33, behenamidopropyl ethyldimonium ethosulfate, oleamidopropyl ethyldimonium ethosulfate, cocamidopropyltrimonium chloride, rinoleamidopropyltrimonium chloride, stearamidopropyl trimonium methosulfate, and mixtures thereof. The amidoamines stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine, palmamidopropyl dimethylamine, Quaternium-33, and behenamidopropyl ethyldimonium ethosulfate are particularly preferred. Stearamidopropyl dimethylamine, cocamidopropyl dimethylamine, isostearamidopropyl dimethylamine, behenamidopropyl dimethylamine, and behenamidopropyl ethyldimonium ethosulfate are highly preferred. Stearamidopropyl dimethylamine is most preferred.

The amidoamines mentioned above may be used individually or in any given combinations with one another, wherein they are included in a total quantity of 0.1 to 5.0% by weight, preferably in a total quantity of 0.2 to 3.0% by weight, and very particularly preferably in a total quantity of 0.5 to 1.5% by weight, in each case based on the weight of the composition according to the invention.

Compositions preferred according to the invention include a mixture of the cationic surfactant types as they are characterized in each case in subclaims 2 and 3, 4 and 5, 6 and 7, and 8 and 9. Particularly preferred compositions according to the invention include one of the following surfactant combinations:
3-behenoyloxy-2-hydroxypropyltrimethylammonium chloride and distearoylethyl hydroxyethylmonium methosulfate;
3-behenoyloxy-2-hydroxypropyltrimethylammonium chloride and dicocoylethyl hydroxyethylmonium methosulfate;
3-Behenoyloxy-2-hydroxypropyltrimethylammonium chloride and Quaternium-87;
3-Behenoyloxy-2-hydroxypropyltrimethylammonium chloride and Quaternium-27;
3-Behenoyloxy-2-hydroxypropyltrimethylammonium chloride and stearamidopropyl dimethylamine;
3-Behenoyloxy-2-hydroxypropyltrimethylammonium chloride and distearoylethyl hydroxyethylmonium methosulfate and Quaternium-87;
3-Behenoyloxy-2-hydroxypropyltrimethylammonium chloride and distearoylethyl hydroxyethylmonium methosulfate and Quaternium-27;
3-Behenoyloxy-2-hydroxypropyltrimethylammonium chloride and distearoylethyl hydroxyethylmonium methosulfate and stearamidopropyl dimethylamine;
3-Behenoyloxy-2-hydroxypropyltrimethylammonium chloride and dicocoylethyl hydroxyethylmonium methosulfate and stearamidopropyl dimethylamine;
3-Behenoyloxy-2-hydroxypropyltrimethylammonium chloride and di stearoylethyl hydroxyethylmonium methosulfate and stearamidopropyl dimethylamine and Quaternium-87;
3-Behenoyloxy-2-hydroxypropyltrimethylammonium chloride and dicocoylethyl hydroxyethylmonium methosulfate and stearamidopropyl dimethylamine and Quaternium-87;
3-Behenoyloxy-2-hydroxypropyltrimethylammonium chloride and distearoylethyl hydroxyethylmonium methosulfate and stearamidopropyl dimethylamine and Quaternium-27;
3-Behenoyloxy-2-hydroxypropyltrimethylammonium chloride and dicocoylethyl hydroxyethylmonium methosulfate and stearamidopropyl dimethylamine and Quaternium-27;
Distearoylethyl hydroxyethylmonium methosulfate and Quaternium-27;
Distearoylethyl hydroxyethylmonium methosulfate and stearamidopropyl dimethylamine;
Dicocoylethyl hydroxyethylmonium methosulfate and stearamidopropyl dimethylamine;
Distearoylethyl hydroxyethylmonium methosulfate and stearamidopropyl dimethylamine and Quaternium-87;
Dicocoylethyl hydroxyethylmonium methosulfate and stearamidopropyl dimethylamine and Quaternium-87;
Distearoylethyl hydroxyethylmonium methosulfate and stearamidopropyl dimethylamine and Quaternium-27;
Dicocoylethyl hydroxyethylmonium methosulfate and stearamidopropyl dimethylamine and Quaternium-27;
Stearamidopropyl dimethylamine and Quaternium-27;
Stearamidopropyl dimethylamine and Quaternium-87.

The second group of ingredients absolutely necessary according to the invention are esters of 3-methyl-1-butanol, also referred to as isoamyl alcohol, with an acid of the general formula R′COOH, where R′ represents a saturated or unsaturated, branched or linear alkyl moiety including 7 to 29, preferably 11 to 21, carbon atoms, and which may include at least one hydroxy group. The stated isoamyl esters are oils having exceptional hair conditioning properties. Isoamyl caprylate, isoamyl caprinate, isoamyl caprate, isoamyl laurate, isoamyl myristate, isoamyl palmitate, isoamyl stearate, isoamyl oleate, isoamyl linolate, isoamyl linoleate, and isoamyl behenate and mixtures of these substances are particularly preferred. Isoamyl laurate is extremely preferred according to the invention.

Compositions particularly preferred according to the invention include at least one ester of 3-methyl-1-butanol (isoamyl alcohol) with an acid of the general formula R′COOH according to claim 1 in a total quantity of 0.1 to 5% by weight, preferably 0.2 to 1% by weight, based on the weight of the composition.

Extremely preferred compositions according to the invention include isoamyl laurate in a total quantity of 0.1 to 5% by weight, preferably 0.2 to 1% by weight, based on the weight of the composition.

To further increase the hair conditioning effect of the combination of the cationic surfactant according to formula (Tkat 1-3) and the isoamyl ester b), the compositions according to the invention optionally include at least one cationic polymer in a total quantity of 0.01 to 5.0% by weight, based on the weight of the composition. Preferred cationic polymers are selected from the following cationic polymers:

Suitable cationic polymers that are derived from natural polymers are cationic derivatives of polysaccharides, for example cationic derivatives of cellulose, starch, or guar. Cationic polysaccharides have the general formula G-O—B—N⁺RₐR_{b}R_{c}A⁻, where

G is an anhydroglucose moiety, for example starch anhydroglucose or cellulose anhydroglucose;
B is a divalent compound group, for example alkylene, oxyalkylene, polyoxyalkylene, or hydroxyalkylene;
Rₐ, R_{b}, and R_{c} are independently alkyl, aryl, alkylaryl, arylalkyl, alkoxyalkyl, or alkoxyaryl, in each case including up to 18 C atoms, wherein the total number of C atoms in Rₐ, R_{b}, and R_{c} is preferably 20 maximum;
A⁻ is a customary counteranion, preferably chloride.

Cationic celluloses are preferred among the polymers which are derived from the natural cationic polymers. Cationic celluloses with different degrees of substitution, cationic charge densities, nitrogen content, and molecular weights are obtainable on the market. In particular, the following cationic celluloses are highly preferred according to the invention.

Polyquaternium-67 is commercially available under the names Polymer SL or Polymer® SK (Amerchol), for example. Another highly preferred cellulose is offered under the trade name Mirustyle® CP from Croda. This is a trimonium and cocodimonium hydroxyethylcellulose as a derivatized cellulose with the INCI name Polyquaternium-72. Polyquaternium-72 may be used in solid form, or also predissolved in aqueous solution.

Examples of suitable cationic polymers that are derived from synthetic polymers are copolymers of

A1) 0.1 to 50%, preferably 10 to 50% (based on the total number of monomers in the copolymer), of monomers of formula (Ia)

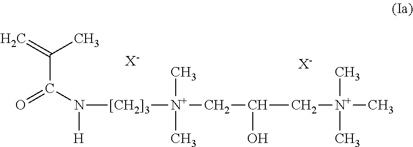

in which
X stands for chloride, sulfate, methosulfate,
A2) monomers from the group comprising acrylic acid, methacrylic acid, and the alkali metal and ammonium salts of these acids,
wherein the monomers A2 constitute 50 to 99.9%, preferably 50 to 90% (based on the total number of monomers in the copolymer), of the copolymer.

Regardless of which of the preferred copolymers A of formula (Ia) is used, hair treatment agents according to the invention are preferred which are characterized in that the ratio (y:z) is 4:1 to 1:2, preferably 4:1 to 1:1.

Regardless of which copolymers A are used in the agents according to the invention, hair treatment agents according to the invention are preferred in which the copolymer A has a molar mass of 10,000 to 20 million gmol⁻¹, preferably 100,000 to 10 million gmol⁻¹, more preferably 500,000 to 5 million gmol⁻¹, and in particular 1.1 million to 2.2 million gmol⁻¹.

A highly preferred copolymer having a structure as described above is commercially available under the name Polyquaternium-74.

Another highly preferred cationic synthetic polymer is a preferably crosslinked homopolymer, poly(methacryloyloxyethyltrimethylammonium chloride), with the INCI name Polyquaternium-37. Such products are commercially available under the names Rheocare® CTH (Cosmetic Rheologies) and Synthalen® CR (3V Sigma), for example.

The homopolymer is preferably used in the form of a nonaqueous polymer dispersion. Such polymer dispersions are commercially available under the names Salcare® SC 95 and Salcare® SC 96.

Hair treatment agents according to the invention, based on their weight, include Polyquaternium-37 in quantities of 0.001 to 5% by weight, preferably 0.0025 to 2.5% by weight, particularly preferably 0.005 to 1% by weight, more preferably 0.0075 to 0.75% by weight, and in particular 0.01 to 0.5% by weight.

Preferred compositions according to the invention may also optionally include at least one sugar surfactant. These sugar-based surfactants are highly preferably used as nonionic surfactants. These are preferably alkyl oligoglycosides or alkenyl oligoglycosides.

These nonionic emulsifiers are known nonionic surfactants according to formula (I)

R¹O-[G]ₚ (I),

in which R¹ stands for an alkyl or alkenyl moiety including 4 to 22 carbon atoms, G stands for a sugar moiety including 5 or 6 carbon atoms, and p stands for numbers from 1 to 10. The alkyl and alkenyl oligoglycosides may be derived from aldoses or ketoses including 5 or 6 carbon atoms, preferably from glucose. The preferred alkyl and/or alkenyl oligoglycosides are thus alkyl and/or alkenyl oligoglucosides. The index number p in general formula (I) indicates the degree of oligomerization (DP), i.e., the distribution of mono- and oligoglycosides, and stands for a number between 1 and 10. While p in the individual molecule must always be an integer and may primarily assume the values p=1 to 6 here, the value p for a specific alkyl oligoglycoside is an analytically determined mathematical value which is usually a fractional number. Alkyl and/or alkenyl oligoglycosides having an average degree of oligomerization p of 1.1 to 3.0 are preferably used. Alkyl and/or alkenyl oligoglycosides having a degree of oligomerization less than 1.7 and in particular between 1.2 and 1.4 are preferred. The alkyl or alkenyl moiety R¹ may be derived from primary alcohols including 4 to 22, preferably 8 to 22, carbon atoms. Typical examples are butanol, caproic alcohol, capryl alcohol, caprinyl alcohol, undecyl alcohol, lauryl alcohol, myristyl alcohol, cetyl alcohol, palmoleyl alcohol, stearyl alcohol, isostearyl alcohol, oleyl alcohol, elaidyl alcohol, petroselinyl alcohol, arachidyl alcohol, gadoleyl alcohol, behenyl alcohol, erucyl alcohol, brassidyl alcohol, natural fatty alcohols such as coco alcohol, and technical mixtures. Examples of commercially available products are the Oramix® types from SEPPIC, for example Oramix® NS 10, and the Plantacare® types, for example Plantacare® 2000UP, Plantacare® 1200UP, Plantacare® 810UP, Plantacare® 818UP.

In addition, the sugar-based emulsifier may be a fatty acid N-alkylpolyhydroxyalkylamide of formula (II)

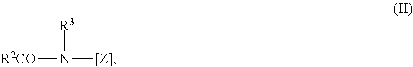

in which R²CO stands for an aliphatic acyl moiety including 6 to 22 carbon atoms, R³ stands for hydrogen or an alkyl or hydroxyalkyl moiety including 1 to 4 carbon atoms, and [Z] stands for a linear or branched polyhydroxyalkyl moiety including 3 to 12 carbon atoms and 3 to 10 hydroxyl groups.

The fatty acid N-alkylpolyhydroxyalkylamides are preferably derived from reducing sugars including 5 or 6 carbon atoms, in particular from glucose. The preferred fatty acid N-alkylpolyhydroxyalkylamides are therefore fatty acid N-alkylglucamides as provided by formula (III):

R²CO—NR³—CH₂—(CHOH)₄CH₂OH (III)

Preferably used as fatty acid N-alkylpolyhydroxyalkylamides are glucamides of formula (III), in which R3 stands for hydrogen or an alkyl group, and R²CO stands for the acyl moiety of caproic acid, caprylic acid, capric acid, lauric acid, myristic acid, palmitic acid, palmitoleic acid, stearic acid, isostearic acid, oleic acid, elaidic acid, petroselinic acid, linoleic acid, linolenic acid, arachidic acid, gadoleic acid, behenic acid, or erucic acid, or the technical mixtures thereof. Furthermore, the polyhydroxyalkylamides may also be derived from maltose and palatinose.

The sugar-based nonionic surfactant is preferably included in the agents used according to the invention in quantities of 0.001-3.0% by weight, based on the overall agent. Quantities of 0.01-2.0% by weight are particularly preferred.

Of course, the compositions according to the invention may optionally include even further customary ingredients in addition to the essential ingredients described above. These are added in particular to impart further desired properties, such as activity against dandruff or increasing the volume, etc., to the compositions. These ingredients are now described.

Saturated, mono- or polyunsaturated, branched or linear fatty alcohols are usable as fatty alcohols (Fatal). Saturated and linear fatty alcohols having a C chain length of C₆-C₁₈, preferably C₈-C₁₈, and very particularly preferably C₁₀-C₁₆, are preferably used. Mono- or polyunsaturated fatty alcohols as well as branched and unsaturated or branched and saturated fatty alcohols having a C chain length of C₆-C₃₀, preferably C₁₀-C₂₂, and very particularly preferably C₁₂-C₂₂, are preferably used. For example, decanol, octanol, octenol, dodecenol, decenol, octadienol, dodecadienol, decadienol, oleyl alcohol, eruca alcohol, ricinol alcohol, lauryl alcohol, myristyl alcohol, capryl alcohol, caprinyl alcohol, linoleyl alcohol, and linolenyl alcohol are usable within the meaning of the invention; this listing is by way of example, and is not intended to be limiting. Fatty alcohol cuts, which are a mixture of different fatty alcohols, are likewise usable according to the invention.

The fatty alcohols are preferably included in a total quantity of 0.1-10% by weight, particularly preferably 2-5% by weight, in each case based on the overall composition.

Linear and/or branched, saturated and/or unsaturated fatty acids including 6-30 carbon atoms may be used as fatty acids (Fatac). Fatty acids including 10-22 carbon atoms are preferred. Mentioned as examples are isostearic acids such as the commercial products Emersol® 871 and Emersol® 875, and isopalmitic acids such as the commercial product Edenor® IP 95, as well as all other fatty acids marketed under the trade names Edenor® (BASF). Other typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, and the technical mixtures thereof. The fatty acid cuts which are obtainable from coconut oil or palm oil are typically particularly preferred; in particular, the use of stearic acid is generally preferred.

The quantity used is 0.1-3% by weight, based on the overall agent. The quantity is preferably 0.5-2% by weight, wherein quantities of 0.8-1% by weight may be very particularly advantageous.

Furthermore, cosmetic oils may be additionally included which are different from the mandatory isoamyl esters. These oil bodies preferably have a melting point less than 50° C., particularly preferably less than 45° C., very particularly preferably less than 40° C., very preferably less than 35° C., and most preferably the cosmetic oils are flowable at a temperature lower than 30° C. These oils are defined and described in greater detail below.

The following are examples of suitable cosmetic oils:

Ester oils having an alcohol component that is different from isoamyl alcohol. Within the meaning of the present patent application, ester oils are understood to mean the esters of polybasic C₆-C₃₀ fatty acids with monohydric or polyhydric C₂-C₃₀ fatty alcohols (not including isoamyl alcohol). The monoesters of fatty acids with monohydric alcohols including 2 to 24 C atoms are preferred. Examples of fatty acid components used in the esters are caproic acid, caprylic acid, 2-ethylhexanoic acid, isononanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, and the technical mixtures thereof. Examples of the fatty alcohol components in the ester oils are isopropyl alcohol, glycerin, caproic alcohol, capryl alcohol, 2-ethylhexyl alcohol, nonyl alcohol, isononyl alcohol, caprinyl alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoleyl alcohol, linolenyl alcohol, eleostearyl alcohol, arachidyl alcohol, gadoleyl alcohol, behenyl alcohol, erucyl alcohol, and brassidyl alcohol, and the technical mixtures thereof. Particularly preferred according to the invention are isopropyl myristate, isononanoic acid-C16-18 alkyl esters, 2-ethylhexyl palmitate, stearic acid-2-ethylhexyl ester, cetyl oleate, glycerin tricaprylate, coco fatty alcohol caprinate/caprylate, n-butyl stearate, oleyl erucate, isopropyl palmitate, oleyl oleate, lauric acid hexyl ester, di-n-butyl adipate, myristyl myristate, cetearyl isononanoate, isononyl isononanoate, and oleic acid decyl ester, and mixtures thereof

Of course, the ester oils may also be alkoxylated with ethylene oxide, propylene oxide, or mixtures of ethylene oxide and propylene oxide. The alkoxylation may take place on the fatty alcohol portion, on the fatty acid portion, or on both portions of the ester oils. However, it is preferred according to the invention that the fatty alcohol has initially been alkoxylated, and subsequently esterified with fatty acid. These compounds are illustrated in a general manner in formula (D4-II):

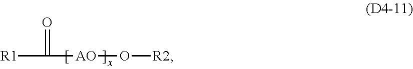

where R1 stands for a saturated or unsaturated, branched or linear, cyclic saturated or cyclic unsaturated acyl moiety including 6 to 30 carbon atoms,
AO stands for ethylene oxide, propylene oxide, or butylene oxide,
X stands for a number between 1 and 200, preferably between 1 and 100, particularly preferably between 1 and 50, very particularly preferably between 1 and 20, very preferably between 1 and 10, and most preferably between 1 and 5,
R2 stands for a saturated or unsaturated, branched or linear, cyclic saturated or cyclic unsaturated alkyl, alkenyl, alkynyl, phenyl, or benzyl moiety including 6 to 30 carbon atoms. Examples of fatty acid components used as moiety R1 in the esters are caproic-acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, and the technical mixtures thereof. Examples of the fatty alcohol components as moiety R2 in the ester oils are benzyl alcohol, isopropyl alcohol, caproic alcohol, capryl alcohol, 2-ethylhexyl alcohol, caprinyl alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, eleostearyl alcohol, arachidyl alcohol, gadoleyl alcohol, behenyl alcohol, erucyl alcohol, and brassidyl alcohol, and the technical mixtures thereof. An ester oil which is particularly preferred according to the invention is available under the INCI name PPG-3 benzyl ether myristate, for example. In addition, ester oils are understood to mean the following:
dicarboxylic acid esters such as di-n-butyl adipate, di-(2-ethylhexyl) adipate, di-(2-ethylhexyl) succinate, and di-isotridecyl azelate, as well as diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate, and
symmetrical, asymmetrical, or cyclic esters of carbonic acid with fatty alcohols, for example glycerin carbonate or dicaprylyl carbonate (Cetiol® CC, for example),
fatty acid partial glycerides, namely, monoglycerides, diglycerides, and the technical mixtures thereof. Typical examples are mono- and/or diglycerides based on caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, eleostearic acid, arachidic acid, gadoleic acid, behenic acid, and erucic acid, and the technical mixtures thereof. Oleic acid monoglycerides are preferably used.
natural oils. Examples of such oils are amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, black currant seed oil, jojoba oil, cocoa butter, linseed oil, macadamia nut oil, corn oil, almond oil, marula oil, evening primrose oil, olive oil, orange oil, palm oil, peach kernel oil, rapeseed oil, rice oil, sea buckthorn berry oil, sea buckthorn seed oil, sesame oil, shea butter, soy oil, sunflower oil, grapeseed oil, walnut oil, wild rose oil, wheat germ oil, and the liquid components of coconut oil. However, other triglyceride oils such as the liquid components of beef tallow and synthetic triglyceride oils are also suitable.
liquid paraffin oils, isoparaffin oils, and synthetic hydrocarbons as well as di-n-alkyl ethers including a total of 12 to 36 C atoms, in particular 12 to 24 C atoms, for example di-n-octyl ether, di-n-decyl ether, di-n-nonyl ether, di-n-undecyl ether, di-n-dodecyl ether, n-hexyl n-octyl ether, n-octyl n-decyl ether, n-decyl n-undecyl ether, n-undecyl n-dodecyl ether, and n-hexyl n-undecyl ether, in addition to di-tert-butyl ether, diisopentyl ether, di-3-ethyldecyl ether, tert-butyl n-octyl ether, isopentyl n-octyl ether, and 2-methyl pentyl-n-octyl ether. The compounds 1,3-di-(2-ethylhexyl) cyclohexane (Cetiol® S) and di-n-octyl ether (Cetiol® OE) which are obtainable as commercial products may be preferred.

Of course, it is also possible according to the invention to use multiple ester oils at the same time. Preferred ester oils are isopropyl myristate, glycerin carbonate, dicaprylyl carbonate, isopropyl palmitate, PPG-3 benzyl ether myristate, cetyl oleate, and oleyl erucate, and mixtures of at least two of these ester oils. Most preferred are mixtures of ester oils when one of the ester oils is dicaprylyl carbonate or isopropyl myristate. Mixtures composed of and including these two ester oils are highly preferred. It is most preferred when a mixture of ester oils including the two latter-named oils is used, when the isopropyl myristate and the caprylyl carbonate are in a ratio of 10:1 to 1:1, preferably 5:1 to 1:1, even more preferably 5:1 to 3:1, in relation to the respective quantities of the ester oils.

The at least one ester oil is included in the agents according to the invention in a total quantity of 0.01 to 7.5% by weight, preferably 0.01 to 5.0% by weight, particularly preferably 0.05 to 5.0% by weight, very preferably 0.2 to 5.0% by weight, based on the weight of the composition according to the invention.

The total quantity is 0.1-10% by weight, preferably 0.1-5% by weight, and particularly preferably 0.1-1% by weight, based on the overall agent.

Examples of emulsifiers that are usable according to the invention are the following:
addition products of 4 to 30 mol ethylene oxide and/or 0 to 5 mol propylene oxide with linear fatty alcohols including 8 to 22 C atoms, with fatty acids including 12 to 22 C atoms, and with alkylphenols including 8 to 15 C atoms in the alkyl group,
C₁₂-C₂₂ fatty acid mono- and diesters of addition products of 1 to 30 mol ethylene oxide with polyols including 3 to 6 carbon atoms, in particular with glycerin,
ethylene oxide and polyglycerin addition products with methylglucoside fatty acid esters, fatty acid alkanolamides,
addition products of 5 to 60 mol ethylene oxide with castor oil and hydrogenated castor oil,
partial esters of polyol including 3-6 carbon atoms with saturated fatty acids including 8 to 22 C atoms,
sterols from animal tissue (zoosterols, cholesterol, lanosterol) and from plant fats (phytosterols, ergosterol, stigmasterol, sitosterol), or from fungi and yeasts (myco sterols),
phospholipids (lecithins, phosphatidylcholines),
fatty acid esters of sugars and sugar alcohols, such as sorbitol,
polyglycerols and polyglycerol derivatives, for example polyglycerol poly-12-hydroxy stearate (commercial product Dehymuls® PGPH).

Further emulsifiers for the compositions according to the invention may be phospholipids. Natural as well as synthetic phospholipids are usable as phospholipid. Natural phospolipids are preferred. In particular compounds of formula (Phosphol-I) are usable as phospholipids.

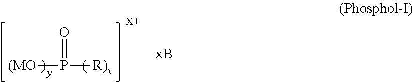

In formula (Phosphol-I), y stands for an integer from 0 to 2, and x stands for an integer from 1 to 3, with the condition that the sum of x and y is equal to 3.

In addition, in the phospolipids of formula (Phospol-I), M stands for hydrogen, an equivalent of an alkali metal or alkaline earth metal cation, an ammonium cation, or an alkyl moiety including 1 to 4 carbon atoms, which is optionally substituted with one or more hydroxy group(s). Compounds in which M stands for a sodium cation are particularly preferred.

Furthermore, in formula (Phospol-I) of the phospolipids to be used according to the invention, B stands for an equivalent of a physiologically compatible anion. Examples of suitable anions are chloride, bromide, iodide, sulfate, perchlorate, tetrafluoroborate, tetraphenylborate, and tetrachlorozincate. The chloride ion is preferred.

In formula (Phospol-I), R stands for a moiety of formula (II)

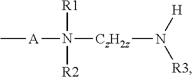

in which z stands for an integer from 1 to 4, in particular 3, and R¹ and R² independently stand for a C₁ to C₄ alkyl moiety which is optionally substituted with one or more hydroxy group(s) or an acyl group.

According to the invention, A stands for one of the units —O—CH₂—CH₂—CH₂—, —O—CH₂—CH₂—, or —O—CH₂—CHOH—CH₂—, with the unit —O—CH₂—CHOH—CH₂— being particularly preferred.

The moiety R³ stands for

(a) a branched or linear, saturated C₈ to C₁₈ acyl moiety or
(b) a branched or linear, mono- or polyunsaturated C₈ to C₁₈ acyl moiety.

Particularly preferred saturated moieties R³ are the moiety stearic acid and the moieties of the mixture of fatty acids obtained from coconut oil.

A particularly preferred unsaturated moiety R³ is the moiety linoleic acid.

Examples of the C₁ to C₄ alkyl groups named as substituents in the compounds according to the invention are the following groups: methyl, ethyl, propyl, isopropyl, and butyl. Ethyl and methyl groups are preferred alkyl groups. Methyl groups are very particularly preferred.

Very particularly preferred phospholipids of formula (Phosphol-I) are the substances known by the INCI names Linoleamidopropyl PG-Dimonium Chloride Phosphate, Cocamidopropyl PG-Dimonium Chloride Phosphate, and Stearamidopropyl PG-Dimonium Chloride Phosphate. These products are marketed, for example, by Mona under the trade names Phospholipid EFA®, Phospholipid PTC®, and Phospholipid SV®.

Furthermore, the glycerophospolipids, which are obtained, for example, as lecithins or phosphatidylcholines, for example from egg yolk or plant seeds, in particular soybeans, are used as phospholipids according to the invention. The phospholipids are in particular phosphoglycerides.

Glycerophospholipids which are particularly suitable according to the invention are obtained from soybeans. Among these, phosphatidylcholines, phosphatidylethanolamines, phosphatidylserines, and phosphatidylinosites as well as mixtures of these substances are particularly preferred.

The particularly preferred phosphatidylcholines have the formula (Phosphol-II)

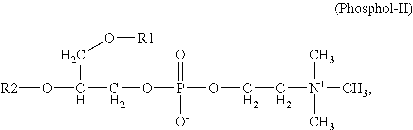

in which the moieties R¹ and R², in each case independently of one another, represent an acyl group composed of fatty acids including 8-30 C atoms, preferably 10-24 C atoms, and particularly preferably 12-22 C atoms. The fatty acid moieties may be saturated as well as mono- or polyunsaturated. The saturated acyl moieties of C₁₂-C₂₂ fatty acids are preferred. The acyl moieties of myristic acid, palmitic acid, stearic acid, arachidic acid, and behenic acid are particularly preferred.

Phosphatidylethanolamines which are preferred according to the invention are those of formula (Phosphol-IIa) or (Phosphol-IIb)

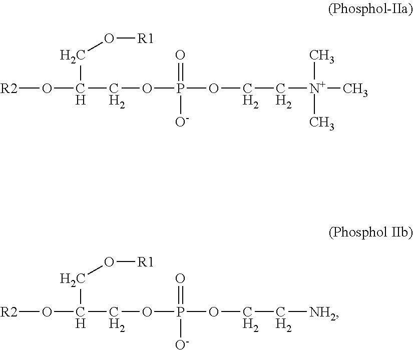

in which the moieties R¹ and R² have the same meanings as in formula (Phosphol-III). Particularly preferred are phosphatidylethanolamines in which R¹ and R² independently represent saturated acyl moieties of fatty acids including 16 or 18 carbon atoms, in particular a palmitoyl or stearoyl moiety.

Phosphatidylserines which are preferred according to the invention are those of structural formula (IIIa) or (IIIb)

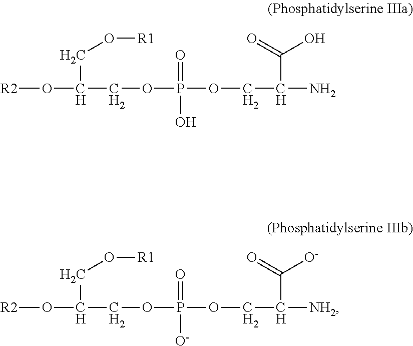

in which R¹ and R² have the same meanings as in formula (Phosphol-I1). Particularly preferred are phosphatidylserines in which R¹ and R² independently represent saturated acyl moieties of fatty acids including 16 or 18 carbon atoms, in particular a palmitoyl or stearoyl moiety.

Phosphatidylinosites which are preferred according to the invention have structural formula (IVa) or (IVb)

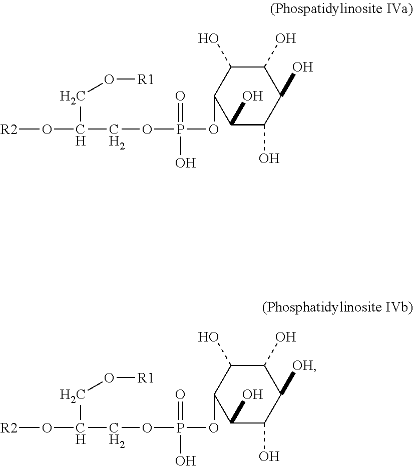

in which the moieties R¹ and R² have the same meanings described for formula (Phosphol-I1). For acyl moieties of palmitic acid, stearic acid, and arachidic acid are preferred; a stearic acid acyl moiety is particularly preferred. R² particularly preferably represents a linear saturated C₂₀ fatty acid acyl moiety (arachoyl moiety).

The glycerophospholipids used according to the invention have an iodine number of 10 maximum, preferably 5 maximum.

According to the invention, it is also possible to use a mixture of multiple phospholipids.

A phospholipid preferred according to the invention is commercially available under the name Emulmetik® 100 (BASF). The phospolipids according to the invention are included in the agents in concentrations of 0.1% by weight to 7.5% by weight, preferably 0.1% by weight to 5% by weight, very particularly preferably in quantities of 0.1% by weight to 3% by weight, and highly preferably in quantities of 0.1 to 1.5% by weight.

The agents according to the invention preferably include the emulsifiers in quantities of 0.1-25% by weight, in particular 0.5-15% by weight, based on the overall agent.

The cationic polymers may be homopolymers or copolymers, the quaternary nitrogen groups being included either in the polymer chain or preferably as a substituent of one or more of the monomers. The monomers which include ammonium groups may be copolymerized with noncationic monomers. Suitable cationic monomers are unsaturated, radically polymerizable compounds which bear at least one cationic group, in particular ammonium-substituted vinyl monomers such as trialkylmethacryloxyalkylammonium, trialkylacryloxyalkylammonium, dialkyldiallylammonium, and quaternary vinyl ammonium monomers having cyclic, cationic nitrogen-including groups such as pyridinium, imidazolium, or quaternary pyrrolidones, for example alkyl vinyl imidazolium, alkyl vinyl pyridinium, or alkyl vinyl pyrrolidone salts. The alkyl groups of these monomers are preferably lower alkyl groups such as C1 to C7 alkyl groups, particularly preferably C1 to C3 alkyl groups.

The monomers including ammonium groups may be copolymerized with noncationic monomers. Examples of suitable comonomers are acrylamide, methacrylamide; alkyl and dialkylacrylamide, alkyl and dialkylmethacrylamide, alkylacrylate, alkylmethacrylate, vinyl caprolactone, vinyl caprolactam, vinylpyrrolidone, vinyl esters such as vinyl acetate; vinyl alcohol, propylene glycol, or ethylene glycol, the alkyl groups of these monomers preferably being C1 to C7 alkyl groups, particularly preferably C1 to C3 alkyl groups.

Suitable polymers including quaternary amine groups are, for example, the polymers described in the CTFA Cosmetic Ingredient Dictionary under the names Polyquaternium, such as methyl vinyl imidazolium chloride/vinylpyrrolidone copolymer (Polyquaternium-16) or quaternized vinylpyrrolidone/dimethylaminoethyl methacrylate copolymer (Polyquaternium-11).

Of the cationic polymers which may be included in the agent according to the invention, for example vinylpyrrolidone/dimethylaminoethyl methacrylate methosulfate copolymer, marketed under the trade names Gafquat® 755 N and Gafquat® 734 by Gaf Co., US, of which Gafquat® 734 is particularly preferred, is suitable. Other cationic polymers are, for example, the copolymer of polyvinylpyrrolidone and imidazolimine marketed, for example, by BASF, Germany under the trade name Luviquat® HM 550, the terpolymer of dimethyldiallylammonium chloride, sodium acrylate, and acrylamide marketed by Calgon, US under the trade name Merquat® Plus 3300, and the vinylpyrrolidone/methacrylic amidopropyltrimethylammonium chloride copolymer marketed by ISP under the trade name Gafquat® HS 100.

Other cationic polymers may include homopolymers of the general formula (P 1),

—{CH₂—[CR¹COO—(CH₂)ₘN⁺R2R3R4]}ₙX⁻,

in which R¹ is —H or —CH₃, R², R³, and R⁴ are independently selected from C1-4 alkyl, alkenyl, or hydroxyalkyl moieties, m=1, 2, 3, or 4, and n is a natural number, and X⁻ is a physiologically compatible organic or inorganic anion. Within the scope of these polymers, preferred according to the invention are those for which at least one of the following conditions applies: R¹ stands for a methyl group, R², R³, and R⁴ stand for methyl groups, and m has the value 2.

Halide ions, sulfate ions, phosphate ions, methosulfate ions, and organic ions such as lactate, citrate, tartrate, and acetate ions, for example, are suitable as physiologically compatible counterions X. Halide ions, in particular chloride, are preferred.

Suitable cationic guar derivatives are marketed under the trade name Jaguar®, and have the INCI name Guar Hydroxypropyltrimonium Chloride. In addition, particularly suitable cationic guar derivatives are also marketed by Hercules under the name N-Hance®. Further cationic guar derivatives are marketed by Cognis under the name Cosmedia®. A preferred cationic guar derivative is the commercial product AquaCat® from Hercules.

A suitable chitosan is marketed, for example, by Kyowa Oil & Fat, Japan, under the trade name Flonac®. A preferred chitosan salt is chitosonium pyrrolidone carboxylate, which is marketed, for example, by Amerchol, US under the name Kytamer® PC. Further chitosan derivatives are readily commercially available under the trade names Hydagen® CMF, Hydagen® HCMF, and Chitolam® NB/101.

Examples of preferred cationic polymers are the following:
cationic alkyl polyglycosides,
cationized honey, for example the commercial product Honeyquat® 50,
polymeric dimethyldiallylammonium salts and the copolymers thereof with esters and amides of acrylic acid and methacrylic acid. The products commercially available under the names Merquat®100 (poly(dimethyldiallylammonium chloride)) and Merquat® 550 (dimethyldiallylammonium chloride-acrylamide copolymer) are examples of such cationic polymers.
vinylpyrrolidone-vinyl imidazolium methochloride copolymers as marketed under the names Luviquat® FC 370, FC 550, FC 905, and HM 552,
quaternized polyvinyl alcohol,
polymers having quaternary nitrogen atoms in the main polymer chain, known under the names Polyquaternium 2, Polyquaternium 17, Polyquaternium 18, and Polyquaternium 27,
vinylpyrrolidone-vinyl caprolactam-acrylate terpolymers, having acrylic acid esters and acrylic acid amides as a third monomer component, commercially available under the name Aquaflex® SF 40, for example.

Likewise usable according to the invention are the copolymers of vinylpyrrolidone, available as the commercial products Copolymer 845 (manufacturer: ISP), Gaffix® VC 713 (manufacturer: ISP), Gafquat®ASCP 1011, Gafquat®HS 110, Luviquat® 8155, and Luviquat® MS 370.

Cationized protein hydrolysates are also included in the cationic polymers, wherein the underlying protein hydrolysate may originate from animals, for example from collagen, milk, or keratin, from plants, for example from wheat, corn, rice, potatoes, soy, or almonds, from marine life forms, for example from fish collagen or algae, or from biotechnology-derived protein hydrolysates. Typical examples of the cationic protein hydrolysates and derivatives according to the invention are the commercially available products listed under the INCI names in the International Cosmetic Ingredient Dictionary and Handbook, (Seventh Edition 1997, The Cosmetic, Toiletry, and Fragrance Association, 1101 17th Street, N.W., Suite 300, Washington, D.C. 20036-4702).

The additional cationic polymers are preferably included in the compositions according to the invention in quantities of 0.01 to 10% by weight, based on the overall agent. Quantities of 0.05 to 5% by weight are particularly preferred.

In addition, amphoteric polymers may be used as polymers. The term “amphoteric polymers” encompasses polymers which include free amino groups as well as free —COOH or SO₃H groups in the molecule and which are capable of forming internal salts; zwitterionic polymers which include quaternary ammonium groups and —COO⁻ or —SO₃⁻ groups in the molecule; and polymers which include —COOH or SO₃H groups and quaternary ammonium groups.

Amphoteric and/or cationic polymers which are preferred according to the invention are those polymers in which a cationic group is derived from at least one of the following monomers:

(i) monomers having quaternary ammonium groups of general formula (Mono1),

R¹—CH═CR²—CO—Z—(CₙH₂ₙ)—N⁽⁺⁾R²R³R⁴A(⁻) (Mono1),
in which R¹ and R² independently stand for hydrogen or a methyl group. and R³, R⁴, and R⁵ independently stand for alkyl groups including 1 to 4 carbon atoms, Z is an NH group or an oxygen atom, n is an integer from 2 to 5, and A⁽⁻⁾ is the anion of an organic or inorganic acid,
   (ii) monomers having quaternary ammonium groups of general formula (Mono2),

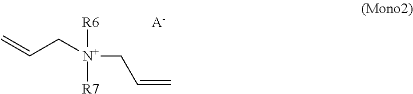

where R⁶ and R⁷ independently stand for a (C₁ to C₄) alkyl group, in particular for a methyl group, and
A⁻ is the anion of an organic or inorganic acid,
   (iii) monomeric carboxylic acids of general formula (Mono3)

R⁸—CH═CR⁹—COOH (Mono3),
in which R⁸ and R⁹, independently of one another, are hydrogen or methyl groups. Polymers are particularly preferred in which monomers of type (i) are used, in which R³, R⁴, and R⁵ are methyl groups, Z is an NH group, and A⁽⁻⁾ is a halide, methoxysulfate, or ethoxysulfate ion; Acrylamidopropyltrimethylammonium chloride is a particularly preferred monomer (i). Acrylic acid is preferably used as monomer (ii) for the stated polymers.
Copolymers of at least one monomer (Mono1) or (Mono2) with the monomer (Mono3), in particular copolymers of monomers (Mono2) and (Mono3) are particularly preferred amphoteric polymers. Copolymers of diallyldimethylammonium chloride and acrylic acid are amphoteric polymers which are very particularly preferably used according to the invention. These copolymers are marketed under the INCI name Polyquaternium-22 under the trade name Merquat® 280 (Nalco), among others.
Furthermore, in addition to a monomer (Mono1) or (Mono2) and a monomer (Mono3), the amphoteric polymers according to the invention may additionally include a monomer (Mono4).
   (iv) monomeric carboxylic acid amides of general formula (Mono4)

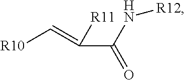

in which R¹⁰ and R¹¹, independently of one another, are hydrogen or methyl groups, and R¹² stands for a hydrogen atom or a (C₁ to C₈) alkyl group.

Amphoteric polymers which are based on a comonomer (Mono4) and very particularly preferably used according to the invention are terpolymers of diallyldimethylammonium chloride, acrylamide, and acrylic acid. These copolymers are marketed under the INCI name Polyquaternium-39 having the trade name Merquat® Plus 3330 (Nalco), among others.

According to the invention, the amphoteric polymers may generally be used directly and also in salt form, which is obtained by neutralizing the polymers with an alkali hydroxide, for example.

The amphoteric polymers are preferably included in the agents according to the invention in quantities of 0.01 to 10% by weight, based on the overall agent. Quantities of 0.01 to 5% by weight are particularly preferred.

The anionic polymers are anionic polymers which have carboxylate and/or sulfonate groups. Examples of anionic monomers of which such polymers may be composed are acrylic acid, methacrylic acid, crotonic acid, maleic acid anhydride, and 2-acrylamido-2-methylpropanesulfonic acid. The acid groups may be present in whole or in part as the sodium, potassium, ammonium, or mono- or triethanolammonium salt. Preferred monomers are 2-acrylamido-2-methylpropanesulfonic acid and acrylic acid.

Anionic polymers which include 2-acrylamido-2-methylpropanesulfonic acid alone or as a comonomer, wherein the sulfonic acid group may be present in whole or in part as the sodium, potassium, ammonium, or mono- or triethanolammonium salt, have proven to be very particularly effective.

The homopolymer of 2-acrylamido-2-methylpropanesulfonic acid, which is commercially available under the name Rheothik® 11-80, for example, is particularly preferred.

Within this embodiment, it may be preferred to use copolymers of at least one anionic monomer and at least one nonionogenic monomer. With regard to the anionic monomers, reference is made to the substances listed above. Preferred nonionogenic monomers are acrylamide, methacrylamide, acrylic acid esters, methacrylic acid esters, vinylpyrrolidone, vinyl ethers, and vinyl esters.

Preferred anionic copolymers are acrylic acid-acrylamide copolymers and in particular polyacrylamide copolymers with monomers including sulfonic acid groups. Such a polymer is included in the commercial product Sepigel® 305 from SEPPIC.

The sodium acryloyldimethyl taurate copolymers, marketed under the name Simulgel®600 as a compound including isohexadecane and Polysorbate-80, have proven to be particularly effective according to the invention.

Likewise preferred anionic homopolymers are uncrosslinked and crosslinked polyacrylic acids. Allyl ethers of pentaerythrite, of sucrose, and of propylene may be preferred crosslinking agents. Such compounds are commercially available under the trademark Carbopol®, for example.

Copolymers of maleic acid anhydride and methyl vinyl ether, in particular those with crosslinking, are likewise color-maintaining polymers. A maleic acid-methyl vinyl ether copolymer crosslinked with 1,9-decadiene is commercially available under the name Stabileze® QM.

The anionic polymers are preferably included in the agents according to the invention in quantities of 0.05 to 10% by weight, based on the overall agent. Quantities of 0.1 to 5% by weight are particularly preferred.

A polyurethane which is very particularly preferred according to the invention is marketed under the trade name Luviset® PUR (BASF).

In another embodiment, the agents according to the invention may include nonionogenic polymers.

Examples of suitable nonionogenic polymers are the following:
vinylpyrrolidone/vinyl ester copolymers, such as those marketed under the trademark Luviskol® (BASF). Luviskol® VA 64 and Luviskol® VA 73, in each case vinylpyrrolidone/vinyl acetate copolymers, are likewise preferred nonionic polymers.
cellulose ethers such as hydroxypropyl cellulose, hydroxyethyl cellulose, and methyl hydroxypropyl cellulose, as marketed, for example, under the trademarks Culminal® and Benecel® (Aqualon) and Natrosol® types (Hercules).
starch and the derivatives thereof, in particular starch ethers such as Structure® XL (National Starch), a multifunctional salt-tolerant starch.
shellac.
polyvinylpyrrolidones, for example those marketed under the name Luviskol® (BASF).

The nonionic polymers are preferably included in the compositions according to the invention in quantities of 0.05 to 10% by weight, based on the overall agent. Quantities of 0.1 to 5% by weight are particularly preferred.

According to the invention, it is also possible for the preparations used to include multiple, in particular two, different polymers of the same charge, and/or in each case one ionic and one amphoteric and/or nonionic polymer.

The polymers (P) are preferably included in the compositions used according to the invention in quantities of 0.01 to 30% by weight, based on the overall composition. Quantities of 0.01 to 25% by weight, in particular 0.01 to 15% by weight, are particularly preferred.

As a further optional ingredient, the agents according to the invention preferably include at least one silicone polymer selected from the group of dimethiconols and/or the group of amino functional silicones and/or the group of dimethicones and/or the group of cyclomethicones. These ingredients are described below.

The dimethicones according to the invention may be linear and branched and cyclic, or cyclic and branched. Linear dimethicones may be represented by the following structural formula (Si1):

(SiR¹₃)—O—(SiR²₂—O—)ₓ—(SiR¹₃) (Si1)

Branched dimethicones may be represented by the structural formula (Sill):

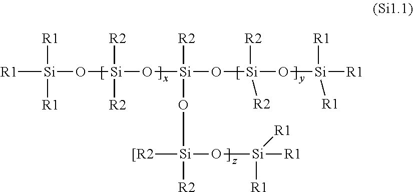

The moieties R¹ and R² independently stand for hydrogen, a methyl moiety, a C2 to C30 linear, saturated or unsaturated hydrocarbon moiety, a phenyl moiety, and/or an aryl moiety. The numbers x, y, and z are integers, and independently have a value of 0 to 50,000. The molecular weights of the dimethicones are between 1000 D and 10,000,000 D. The viscosities are between 100 and 10,000,000 cPs, measured at 25° C. using a glass capillary viscosimeter according to Dow Corning Corporate Test Method CTM 0004, Jul. 20, 1970. Preferred viscosities are between 1000 and 5,000,000 cPs, and very particularly preferred viscosities are between 10,000 and 3,000,000 cPs. The most preferred range is between 50,000 and 2,000,000 cPs. Viscosities around the range of approximately 60,000 cPs are highly preferred. Reference is made to the product “Dow Corning 200 with 60,000 cSt” as an example.

Particularly preferred cosmetic or dermatological preparations according to the invention are characterized in that they include at least one silicone of formula (Si1.2)

(CH₃)₃Si—[O—Si(CH₃)₂]ₓ—O—Si(CH₃)₃ (Si1.2),

in which x stands for a number from 0 to 100, preferably 0 to 50, more preferably 0 to 20, and in particular 0 to 10.

The dimethicones (Si1) are included in the compositions according to the invention in quantities of 0.01 to 10% by weight, preferably 0.01 to 8% by weight, particularly preferably 0.1 to 7.5% by weight, and in particular 0.1 to 5% by weight, based on the overall composition.

Particularly preferred agents according to the invention include one or more amino functional silicones. Such silicones may be described, for example, by formula (Si-2)

M(RₐQ_{b}SiO_{(4-a-b)/2})ₓ(R_{c}SiO_{(4-c)/2})_{y}M (Si-2),

where in the above formula
R is a hydrocarbon or a hydrocarbon moiety including 1 to approximately 6 carbon atoms,
Q is a polar moiety of general formula —R′HZ,
   where
   R¹ is a divalent connecting group which is bound to hydrogen and the moiety Z, and composed of carbon and hydrogen atoms, carbon, hydrogen, and oxygen atoms, or carbon, hydrogen, and nitrogen atoms, and
   Z is an organic amino functional moiety including at least one amino functional group;
a assumes values in the range of approximately 0 to approximately 2,
b assumes values in the range of approximately 1 to approximately 3,
a+b is less than or equal to 3,
c is a number in the range of approximately 1 to approximately 3,
x is a number in the range of 1 to approximately 2000, preferably from approximately 3 to approximately 50, and most preferably from approximately 3 to approximately 25,
y is a number in the range of approximately 20 to approximately 10,000, preferably from approximately 125 to approximately 10,000, and most preferably from approximately 150 to approximately 1.000, and
M is a suitable silicone end group as known in the prior art, preferably trimethylsiloxy.

Z according to formula (Si-2) is an organic amino functional moiety including at least one functional amino group. One possible formula for Z is NH(CH₂)_{z}NH₂, where z is an integer greater than or equal to 1. Another possible formula for Z is —NH(CH₂)_{z}(CH₂)_{zz}NH, where both z and zz, independently from one another, are an integer greater than or equal to 1; this structure includes diamino ring structures such as piperazinyl. Z is most preferably an —NHCH₂CH₂NH₂ moiety. Another possible formula for Z is —NH(CH₂)_{z}(CH₂)_{zz}NX₂ or —NX₂, where each X of X₂ is independently selected from the group comprising hydrogen and alkyl groups including 1 to 12 carbon atoms, and zz is 0.

Q according to formula (Si-2) is most preferably a polar amino functional moiety of formula —CH₂CH₂CH₂NHCH₂CH₂NH₂.

In formula (Si-2), a assumes values in the range of 0 to 2, b assumes values in the range of 2 to 3, a+b is less than or equal to 3, and c is a number in the range of 1 to 3.

Cationic silicone oils such as the commercially available products Dow Corning (DC) 929 Emulsion, DC 2-2078, DC 5-7113, SM-2059 (General Electric), and SLM-55067 (Wacker) are suitable according to the invention.

Particularly preferred agents according to the invention are characterized in that they include at least one amino functional silicone of formula (Si3-a)

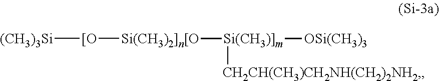

where m and n are numbers whose sum (m+n) is between 1 and 2000, preferably between 50 and 150, n preferably assuming values from 0 to 1999 and in particular from 49 to 149, and m preferably assuming values from 1 to 2000, in particular from 1 to 10.

These silicones are designated as Trimethylsilylamodimethicone according to the INCI declaration, and are obtainable, for example, under the name Q2-7224 (manufacturer: Dow Corning; a stabilized trimethylsilylamodimethicone).

Also particularly preferred are agents according to the invention which include at least one amino functional silicone of formula (Si-3b)

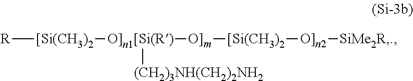

where
R stands for —OH, an (optionally ethoxylated and/or propoxylated) (C₁ to C₂₀) alkoxy group or a —CH₃ group,
R1 stands for —OH, a (C₁ to C₂₀) alkoxy group, or a —CH₃ group, and
m, n1, and n2 are numbers whose sum (m+n1+n2) is between 1 and 2000, preferably between 50 and 150, the sum (n1+n2) preferably assuming values from 0 to 1999 and in particular from 49 to 149, and m preferably assuming values from 1 to 2000, in particular from 1 to 10.

According to the INCI declaration, these silicones are designated as Amodimethicone or as functionalized Amodimethicone, for example Bis(C13-15 alkoxy) PG Amodimethicone (obtainable, for example, as the commercial product DC 8500 from Dow Corning), Trideceth-9 PG-Amodimethicone (obtainable, for example, as the commercial product Silcare Silicone SEA from Clariant).

Suitable diquaternary silicones are selected from compounds of general formula (Si3c))

[R¹R²R³N⁺-A-SiR⁷R⁸—(O—SiR⁹R¹⁰)ₙ—O—SiR¹¹R¹²-A-N⁺R⁴R⁵R⁶]2X⁻ (Si3c),

where the moieties R1 to R6 independently stand for C1 to C22 alkyl moieties which may include hydroxy groups, and wherein preferably at least one of the moieties includes at least 8 C atoms and the other moieties include 1 to 4 C atoms,
the moieties R7 to R12, independently of one another, are the same or different and mean
C1 to C10 alkyl or phenyl,
A means a divalent organic connecting group,
n is a number from 0 to 200, preferably from 10 to 120, particularly preferably from 10 to 40, and
X⁻ is an anion.

The divalent connecting group is preferably a C1 to C12 alkylene or alkoxyalkylene group, which may be substituted with one or more hydroxyl groups. The —(CH₂)₃—O—CH₂—CH(OH)—CH₂— group is particularly preferred.

The anion X⁻ may be a halide ion, an acetate, an organic carboxylate, or a compound of general formula RSO₃⁻, where R has the meaning of C1 to C4 alkyl moieties.

A preferred diquaternary silicone has the general formula (Si3d)

[RN⁺Me₂-A-(SiMe₂O)ₙ—SiMe₂-A-N⁺Me₂R]2CH₃COO⁻ (Si3d),

where A is the —(CH₂)₃—O—CH₂—CH(OH)—CH₂— group,

R is an alkyl moiety including at least 8 C atoms, and

n is a number from 10 to 120.

Suitable silicone polymers having two terminal quaternary ammonium groups are known under the INCI name Quaternium-80. These are dimethylsiloxanes with two terminal trialkylammonium groups. Such diquaternary polydimethylsiloxanes are marketed by Evonik under the trade names Abil® Quat 3270, 3272, and 3474.

Cosmetic or dermatological preparations which are preferred according to the invention are characterized in that, based on their weight, they include 0.01 to 10% by weight, preferably 0.01 to 8% by weight, particularly preferably 0.1 to 7.5% by weight, and in particular 0.2 to 5% by weight, of amino functional silicone(s) and/or diquaternary silicone.

The compositions according to the invention may include at least one polyammonium polysiloxane compound as silicone. The polyammonium polysiloxane compounds may be obtained, for example, from GE Bayer Silicones under the trade name Baysilone®. The products having the names Baysilone TP 3911, SME 253, and SFE 839 are preferred. Use of Baysilone TP 3911 as the active component of the compositions according to the invention is very particularly preferred. The polyammonium polysiloxane compounds are included in the compositions according to the invention in a quantity of 0.01 to 10% by weight, preferably 0.01 to 7.5% by weight, particularly preferably 0.01 to 5.0% by weight, very particularly preferably 0.05 to 2.5% by weight, in each case based on the overall composition.

Furthermore, even further cationic amino functional silicone polymers may be used. EP 1887024 A1 describes novel cationic amino functional silicones which in particular enhance the luster in agents for care of surfaces, for example human hair. These cationic silicone polymers are characterized in that they have a silicone backbone structure and at least one polyether portion and also at least one portion having an ammonium structure. Examples of preferred cationic silicone polymers within the meaning of the present invention are in particular the compounds of above-referenced EP 1887024 A1, in particular the compounds having the INCI names: Silicone Quaternium-1, Silicone Quaternium-2, Silicone Quaternium-3, Silicone Quaternium-4, Silicone Quaternium-5, Silicone Quaternium-6, Silicone Quaternium-7, Silicone Quaternium-8, Silicone Quaternium-9, Silicone Quaternium-10, Silicone Quaternium-11, Silicone Quaternium-12, Silicone Quaternium-15, Silicone Quaternium-16, Silicone Quaternium-17, Silicone Quaternium-18, Silicone Quaternium-20, Silicone Quaternium-21, Silicone Quaternium-22, and Silicone Quaternium-2 Panthenol Succinate and Silicone Quaternium-16/Glycidyl Dimethicone Crosspolymer. In particular Silicone Quaternium-22 is most preferred. This raw material is marketed, for example, by Evonik under the trade name Abil® T-Quat 60.

The above-mentioned cationic amino functional silicone polymers are included in the compositions according to the invention in quantities of 0.01 to 20% by weight, preferably in quantities of 0.05 to 10% by weight, and very particularly preferably in quantities of 0.1 to 7.5% by weight. The very best results are obtained using quantities of 0.1 to 5% by weight, in each case based on the overall composition of the particular agent. It may be particularly advantageous according to the invention when solely the above-mentioned silicones are used as silicones.

The cyclic dimethicones designated as Cyclomethicone according to INCI are also preferably usable according to the invention. Cosmetic or dermatological preparations according to the invention are preferred here which include at least one silicone of formula (Si-4)

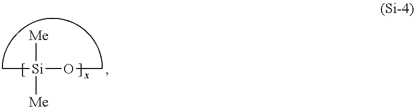

in which x stands for a number from 3 to 200, preferably from 3 to 10, more preferably from 3 to 7, and in particular 3, 4, 5, or 6.

Agents which are likewise preferred according to the invention are characterized in that they include at least one silicone of formula (Si-5)

R3Si—[O—SiR₂]ₓ—(CH₂)ₙ—[O—SIR₂]_{y}—O—SiR₃ (Si-5),

in which R stands for the same or different moieties from the group H, phenyl, benzyl, and —CH₂—CH(CH₃)Ph, C₁₋₂₀ alkyl moieties, preferably —CH₃, —CH₂CH₃, —CH₂CH₂CH₃, —CH(CH₃)₂, CH₂CH₂CH₂CH₃, —CH₂CH(CH₃)₂, —CH(CH₃)CH₂CH₃, or —C(CH₃)₃, x and y each stand for a number from 0 to 200, preferably from 0 to 10, more preferably from 0 to 7, and in particular 0, 1, 2, 3, 4, 5, or 6, and n stands for a number from 0 to 10, preferably from 1 to 8, and in particular 2, 3, 4, 5, 6.

In addition to the dimethicones, dimethiconols, amodimethicones, and/or cyclomethicones, water-soluble silicones may be included as further silicones in the compositions according to the invention.

Corresponding hydrophilic silicones are selected, for example, from the compounds of formula(s) (Si-6) and/or (Si-7). In particular, preferred water-soluble surfactants based on silicone are selected from the group of dimethicone copolyols, which are preferably alkoxylated, in particular polyethoxylated or polypropoxylated.

According to the invention, dimethicone copolyols are preferably understood to mean polyoxyalkylene-modified dimethylpolysiloxanes of general formulas (Si-6) or (Si-7):

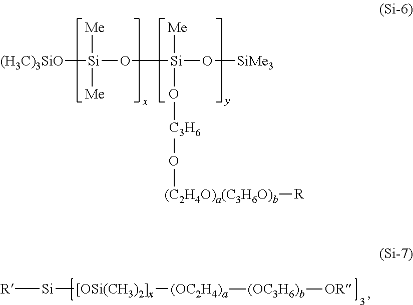

where the moiety R stands for a hydrogen atom, an alkyl group including 1 to 12 C atoms, an alkoxy group including 1 to 12 C atoms, or a hydroxyl group, the moieties R′ and R″ mean alkyl groups including 1 to 12 C atoms, x stands for an integer from 1 to 100, preferably from 20 to 30, y stands for an integer from 1 to 20, preferably from 2 to 10, and a and b stand for integers from 0 to 50, preferably from 10 to 30.

Examples of particularly preferred dimethicone copolyols within the meaning of the invention are the products which are marketed by Union Carbide Corporation under the trade name Silwet, and by Dow Corning. Dow Corning 190 and Dow Corning 193 are dimethicone copolyols that are particularly preferred according to the invention.

The dimethicone copolyols are included in the compositions according to the invention in quantities of 0.01 to 10% by weight, preferably 0.01 to 8% by weight, particularly preferably 0.1 to 7.5% by weight, and in particular 0.1 to 5% by weight, of dimethicone copolyol, based on the composition.

Lastly, the silicone compounds are understood to include dimethiconols (Si8). The dimethiconols preferred according to the invention may be both linear and branched, as well as cyclic or cyclic and branched. Linear dimethiconols may be represented by the following structural formula (Si8-I):

(SiOHR¹₂)—O—(SiR²₂—O—)ₓ—(SiOHR¹₂) (Si8-I)

Branched dimethiconols may be represented by the structural formula (Si8-II):

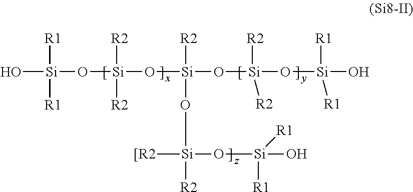

The moieties R¹ and R² independently stand for hydrogen, a methyl moiety, a C2 to C30 linear, saturated or unsaturated hydrocarbon moiety, a phenyl moiety, and/or an aryl moiety. The numbers x, y, and z are integers, and independently have a value from 0 to 50,000. The molecular weights of the dimethicones are between 1000 D and 10,000,000 D. The viscosities are between 100 and 10,000,000 cPs, measured at 25° C. using a glass capillary viscosimeter according to Dow Corning Corporate Test Method CTM 0004, Jul. 20, 1970. Preferred viscosities are between 1000 and 5,000,000 cPs, and very particularly preferred viscosities are between 10,000 and 3,000,000 cPs. The most preferred range is between 50,000 and 2,000,000 cPs.

The following commercial products are mentioned as examples of these types of products: Dow Corning 1-1254 Fluid, Dow Corning 2-9023 Fluid, Dow Corning 2-9026 Fluid, Abil OSW 5 (Degussa Care Specialties), Dow Corning 1401 Fluid, Dow Corning 1403 Fluid, Dow Corning 1501 Fluid, Dow Corning 1784 HVF Emulsion, Dow Corning 9546 Silicone Elastomer Blend, SM555, SM2725, SM2765, SM2785 (the four latter-named products are GE Silicones), and Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, and Wacker-Belsil DM 60081 VP (all from Wacker-Chemie GmbH).

The dimethiconols (Si8) are included in the compositions according to the invention in quantities of 0.01 to 10% by weight, preferably 0.01 to 8% by weight, particularly preferably 0.1 to 7.5% by weight, and in particular 0.1 to 5% by weight of dimethiconol, based on the composition.

A further synergistic active substance according to the invention in the compositions according to the invention including the active substance combination according to the invention are protein hydrolysates and/or the derivatives thereof (P).

Protein hydrolysates of plant, animal, or marine origin or of synthetic origin may be used according to the invention.

Examples of animal protein hydrolysates are elastin, collagen, keratin, silk, and milk protein hydrolysates, which may also be present in the form of salts.

Also preferred according to the invention are plant protein hydrolysates such as soy, almond, pea, moringa, potato, and wheat protein hydrolysates. Such products are obtainable, for example, under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda), Crotein® (Croda) and Puricare® LS 9658 from Laboratoires Serobiologiques.

Further protein hydrolysates which are preferred according to the invention are of marine origin. These include, for example, collagen hydrolysates from fish or algae, and protein hydrolysates from mussels or pearl hydrolysates. Examples of pearl extracts according to the invention are the commercial products Pearl Protein Extract BG® or Crodarom® Pearl.

The protein hydrolysates (P) are included in the compositions in concentrations of 0.01% by weight to 20% by weight, preferably 0.05% by weight to 15% by weight, and very particularly preferably in quantities of 0.05% by weight to 5% by weight.

The activity of the compositions according to the invention may be further increased by 2-pyrrolidinone-5-carboxylic acid and the derivatives thereof (J). The sodium, potassium, calcium, magnesium, or ammonium salts, in which the ammonium ion bears one to three C₁ to C₄ alkyl groups in addition to hydrogen, are preferred. The sodium salt is very particularly preferred. The quantities used in the agents according to the invention are 0.05 to 10% by weight, particularly preferably 0.1 to 5% by weight, and in particular 0.1 to 3% by weight, based on the overall agent.

Another preferred group of ingredients in the compositions according to the invention including the active substance combination according to the invention are vitamins, provitamins, or vitamin precursors.

Vitamins, provitamins, and vitamin precursors are preferred which are associated with the groups A, B, C, E, F, and H.

The group of substances denoted as vitamin A includes retinol (vitamin A₁) and 3,4-didehydroretinol (vitamin A2). β-Carotene is the provitamin of retinol. For example, vitamin A acid and the esters thereof, vitamin A aldehyde, and vitamin A alcohol and the esters thereof, such as the palmitate and the acetate, are suitable as the vitamin A component. The agents according to the invention preferably include the vitamin A component in quantities of 0.05-1% by weight, based on the overall preparation.

The Vitamin B group or the Vitamin B complex includes the following, among others: Vitamin B₁ (thiamin), Vitamin B₂ (riboflavin), Vitamin B₃: the compounds nicotinic acid and nicotinic acid amide (niacinamide) are frequently referred to by this name. Nicotinic acid amide, which is preferably included in the agents used according to the invention in quantities of 0.05 to 1% by weight, based on the overall agent, is preferred according to the invention; Vitamin B5 (pantothenic acid, panthenol, and pantolactone). Within this group, panthenol and/or pantolactone is/are preferably used. Derivatives of panthenol which are usable according to the invention are in particular the esters and ethers of panthenol and cationically derivatized panthenols. Examples of individual representatives are panthenol triacetate, panthenol monoethyl ether and the monoacetate thereof, and cationic panthenol derivatives. Pantothenic acid is preferably used in the present invention as the derivative in the form of the more stable calcium salts and sodium salts (Ca pantothenate, Na pantothenate); Vitamin B6 (pyridoxine as well as pyridoxamine and pyridoxal).

The stated compounds of the vitamin B type, in particular vitamins B3, B5, and B6, are preferably included in the agents according to the invention in quantities of 0.05-10% by weight, based on the overall agent. Quantities of 0.1-5% by weight are particularly preferred.

Vitamin C (ascorbic acid). Vitamin C is preferably used in the agents according to the invention in quantities of 0.1 to 3% by weight, based on the overall agent. Use in the form of the palmitic acid ester, the glucosides, or phosphates may be preferred. Use in combination with tocopherols may likewise be preferred.

Vitamin E (tocopherols, in particular α-tocopherol). Tocopherol and its derivatives, which include in particular the esters such as the acetate, the nicotinate, the phosphate, and the succinate, are preferably included in the agents according to the invention in quantities of 0.05-1% by weight, based on the overall agent.

Vitamin F. The term “Vitamin F” is customarily understood as essential fatty acids, in particular linoleic acid, linolenic acid, and arachidonic acid.

Vitamin H. The compound (3aS,4S,6aR)-2-oxohexahydrothienol[3,4-d]imidazole-4-valeric acid is referred to as vitamin H, which has become known in common usage as biotin. Biotin is preferably included in the agents according to the invention in quantities of 0.0001 to 1.0% by weight, in particular in quantities of 0.001 to 0.01% by weight.

The compositions according to the invention preferably include vitamins, provitamins, and vitamin precursors from the groups A, B, E, and H. Panthenol, pantolactone, pyridoxine, and the derivatives thereof, as well as nicotinic acid amide and biotin, are particularly preferred.

A particularly preferred group of ingredients in the cosmetic compositions according to the invention are the following betaines: carnitine, carnitine tartrate, carnitine magnesium citrate, acetylcarnitine, betalains, 1,1-dimethylproline, choline, choline chloride, choline bitartrate, choline dihydrogen citrate, and the compound N,N,N-trimethylglycine, which is referred to in the literature as betaine.

Carnitine, histidine, choline, and betaine are preferably used. L-Carnitine tartrate is used in one particularly preferred embodiment of the invention.

Taurine and/or a derivative of taurine is a particularly important ingredient. Taurine is understood to mean solely 2-aminoethansulfonic acid, and a derivative is understood to mean the explicitly mentioned derivatives of taurine. The derivatives of taurine are understood to be N-monomethyltaurine, N,N-dimethyltaurine, taurine lysylate, taurine tartrate, taurine ornithate, lysyl taurine, and ornithyl taurine. Further taurine derivatives within the meaning of the present invention are taurocholic acid and hypotaurine.

Agents according to the invention are particularly preferred which, based on their weight, include 0.0001 to 10.0% by weight, preferably 0.0005 to 5.0% by weight, particularly preferably 0.001 to 2.0% by weight, and in particular 0.001 to 1.0% by weight, of taurine and/or a derivative of taurine.

In another embodiment which is preferred according to the invention, the compositions according to the invention include bioquinones. In the agents according to the invention, one or more ubiquinone(s) and/or plastoquinone(s) is/are understood to be suitable bioquinones. Ubiquinones preferred according to the invention have the following formula:

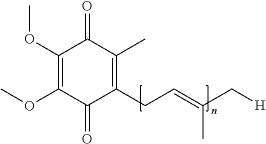

where n=6, 7, 8, 9, or 10.

Coenzyme Q-10 is most preferred.

Preferred compositions according to the invention include purine and/or purine derivatives in narrow quantity ranges. Here, cosmetic agents preferred according to the invention are characterized in that they include, based on their weight, 0.001 to 2.5% by weight, preferably 0.0025 to 1% by weight, particularly preferably 0.005 to 0.5% by weight, and in particular 0.01 to 0.1% by weight, of purine(s) and/or purine derivative(s). Cosmetic agents preferred according to the invention are characterized in that they include purine, adenine, guanine, uric acid, hypoxanthine, 6-purinethiol, 6-thioguanine, xanthine, caffeine, theobromine, or theophylline. Caffeine is most preferred in hair cosmetic preparations.

In another preferred embodiment of the present invention, the cosmetic agent includes ectoine ((S)-2-methyl-1,4,5,6-tetrahydro-4-pyrimidine carboxylic acid).

Agents are particularly preferred according to the invention which, based on their weight, include 0.00001 to 10.0% by weight, preferably 0.0001 to 5.0% by weight, and in particular 0.001 to 3% by weight, of the active substances from the group comprising carnitine, taurine, coenzyme Q-10, ectoine, a vitamin of the B series, a purine and the derivatives thereof, or physiologically acceptable salts.

In another embodiment, the agents according to the invention should additionally include at least one UV light protection filter. UVB filters may be oil-soluble or water-soluble.

The following are mentioned as examples of oil-soluble substances:
3-benzylidene camphor, for example 3-(4-methylbenzylidene) camphor;
4-aminobenzoic acid derivatives, preferably 4-(dimethylamino)benzoic acid-2-ethylhexyl ester, 4-(dimethylamino)benzoic acid-2-octyl ester, and 4-(dimethylamino)benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, and 2-cyano-3-phenylcinnamic acid-2-ethylhexyl ester (octocrylene);
esters of salicylic acid, preferably salicylic acid-2-ethylhexyl ester, salicylic acid-4-isopropylbenzyl ester, and salicylic acid homomenthyl ester;
derivatives of benzophenone, preferably 2-hydroxy-4-methoxybenzophenone, 2-hydroxy-4-methoxy-4 ‘-methylbenzophenone, and 2,2’-dihydroxy-4-methoxybenzophenone;
esters of benzalmalonic acid, preferably 4-methoxybenzalmalonic acid di-2-ethylhexyl ester;
triazine derivatives such as 2,4, 6-trianilino-(p-carbo-2′-ethyl-1′-hexyloxy)-1,3,5-triazine and octyl triazone;
propane-1,3-diones such as 1-(4-tert-butylphenyl)-3-(4′-methoxyphenyl)propane-1,3-dione.

The following are suitable water-soluble substances:
2-phenylbenzimidazole-5-sulfonic acid and the alkali, alkaline earth, ammonium, alkylammonium, alkanolammonium, and glucammonium salts thereof;
sulfonic acid derivatives of benzophenones, preferably 2-hydroxy-4-methoxybenzophenone-5-sulfonic acid and the salts thereof;
sulfonic acid derivatives of 3-benzylidene camphor, such as 4-(2-oxo-3-bornylidenemethyl)benzenesulfonic acid and 2-methyl-5-(2-oxo-3-bornylidene)sulfonic acid and the salts thereof

In particular derivatives of benzoyl methane, such as 1-(4′-tert-butylphenyl)-3-(4 ‘-methoxyphenyl)propane-1,3-dione or 1-phenyl-3-(4’-isopropylphenyl)propane-1,3-dione, are suitable as typical UV-A filters. The UV-A and UV-B filters may of course also be used in mixtures. In addition to the mentioned soluble substances, also suitable for this purpose are insoluble pigments, in particular finely dispersed metal oxides or salts, such as titanium dioxide, zinc oxide, iron oxide, aluminum oxide, cerium oxide, zirconium oxide, silicates, in particular talc, barium sulfate, and zinc stearate. The particles should have an average diameter of less than 100 nm, preferably between 5 and 50 nm, and in particular between 15 and 30 nm. They may have a spherical shape, although particles having a shape that is ellipsoidal or otherwise different from a spherical shape may also be used.

Lastly, further advantages result from the use of plant extracts (L) in the compositions according to the invention. Primarily preferred according to the invention are extracts of green tea, oak bark, nettle, witch hazel, hops, henna, chamomile, burdock root, horsetail, hawthorn, lime blossom, almond, aloe vera, spruce needle, horse chestnut, sandalwood, juniper berry, coconut, mango, apricot, lemon, wheat, kiwi fruit, melon, orange, grapefruit, sage, rosemary, birch, mallow, valerian, cuckoo flower, wild thyme, yarrow, thyme, lemon balm, restharrow, coltsfoot, marsh mallow, meristem, ginseng, coffee, cacao, moringa, ginger root, and Ayurvedic plant extracts such as Aegle marmelos (bilwa), Cyperus rotundus (nagarmotha), Emblica officinalis (amalaki), Morinda citrifolia (ashyuka), Tinospora cordifolia (guduchi), Santalum album (chandana), Crocus sativus (kumkuma), Cinnamomum zeylanicum, and Nelumbo nucifera (kamala), sweet grasses such as wheat, barley, rye, oats, spelt, corn, the various varieties of millet (proso millet, crabgrass, foxtail millet as examples), sugar cane, ryegrass, meadow foxtail, oatgrass, bentgrass, meadow fescue, moor grass, bamboo, cotton grass, fountain grasses, Andropogoneae (Imperata cylindrica, also referred to as flame grass or cogon grass), buffalo grass, cordgrasses, dog's tooth grasses, lovegrasses, Cymbopogon (lemongrass), Oryzeae (rice), Zizania (wild rice), beachgrass, perennial oatgrass, soft grasses, quaking grasses, poa grasses, wild rye, and Echinacea, in particular Echinacea angustifolia DC, Echinacea paradoxa (Norton), Echinacea simulata, E. atrorubens, E. tennesiensis, Echinacea strigosa (Mc Gregor), Echinacea laevigata, Echinacea purpurea (L.) Moench, and Echinacea pallida (Nutt), all types of wine, and the pericarp of Litchi chinensis.

The plant extracts may be used according to the invention in pure or diluted form. If they are used in diluted form, they typically include approximately 2-80% by weight of active substance, and as solvent, the extraction agent or extraction agent mixture used in their production.

In addition, the cosmetic agents may include further active substances, auxiliary substances, and additives, for example acidifiers such as citric acid and lactic acid, dimethyl isosorbide, and cyclodextrins, dyes for coloring the agent, antidandruff active substances such as piroctone olamine, zinc omadine, and climbazole, complexing agents such as EDTA, NTA, β-alaninediacetic acid, and phosphonic acids, opacifiers such as latex, and styrene/PVP and styrene/acrylamide copolymers, pearlescent agents such as ethylene glycol mono- and distearate and PEG-3 distearate, pigments, stabilizing agents for hydrogen peroxide and other oxidizing agents, propellants such as propane-butane mixtures, N20, dimethyl ether, CO₂, and air, antioxidants, fragrance oils, odorants, and fragrance ingredients.

With regard to further optional components and the quantities of these components used, express reference is made to the relevant handbooks known to those skilled in the art.

As previously mentioned, particular importance is attached to the high care effect of the agents according to the invention.

A further subject matter of the invention relates to the use of a composition according to the invention or preferred according to the invention for improving the resistance of the surface of keratin fibers to physical damage. According to the invention, physical damage is understood to mean the effect of UV light, the effect of heat during blow drying, and mechanical effects in the combing and brushing of the hair.

A further subject matter of the invention relates to the use of a composition according to the invention or preferred according to the invention for improving the resistance to washout of the color of dyed keratin fibers.

The statements made with regard to the agents according to the invention also apply mutatis mutandis with regard to further preferred embodiments of the uses according to the invention.

A further subject matter of the invention therefore relates to a method for hair treatment, in which a cosmetic agent according to claim 1 is applied to the hair, and rinsed from the hair after an exposure period. The exposure period is preferably 5 seconds to 100 minutes, particularly preferably 1 to 45 minutes, and very particularly preferably 2 to 30 minutes.

The invention also encompasses a method in which a cosmetic agent according to claim 1 is applied to the hair and remains there. According to the invention, “remaining on the hair” is understood to mean that the agent is not rinsed from the hair immediately after being applied. Instead, in this case the agent remains on the hair for more than 100 minutes and until the next hair washing.

The statements made with regard to the agents according to the invention also apply mutatis mutandis with regard to further preferred embodiments of the methods according to the invention.

### Examples

All stated quantities are in weight-%. The following hair conditioner formulation was prepared using known production methods:

Ingredient

| | |
|---|---|
| Cetearyl alcohol | 4.5 |
| Distearoylethyl hydroxyethylmonium methosulfate | 1.5 |
| Glyceryl mono/distearate | 0.7 |
| 3-Behenoyloxy-2-hydroxypropyltrimethylammonium chloride | 3.0 |
| 2-Methyl-2,4-pentanediol | 1.0 |
| Stearamidopropyl dimethylamine | 0.4 |
| Cocoa butter | 0.6 |
| Citric acid | 0.7 |
| Methylparaben Na salt | 0.3 |
| Panthenol | 0.2 |
| Isoamyl laurate | 2.0 |
| Keratin hydrolysate | 0.05 |
| Phenoxyethanol | 0.4 |
| Fragrance | 0.3 |
| Water | ad 100 |

The two hair conditioner formulations given below (according to the invention, comparative formulation) were used on five individuals in the half-side test, and their application properties were rated by two trained hairdressers.

For this purpose, the hair of the test subjects was initially washed with a shampoo. The hair was then parted; on one half of the head, the hair was treated with the conditioner according to the invention, and on the other half of the head the hair was treated with the conditioner not according to the invention.

The hair was then rinsed with water and dried, either with a blow dryer or a dryer hood.

| | Hair conditioner | Hair conditioner. |
|---|---|---|
| | according to the | comparative |
| | invention | formulation |
| Dehyquart F75 | 1.5 | 1.5 |
| Isoamyl laurate | 1.0 | — |
| Cutina GMS V | 0.7 | 0.7 |
| Cetearyl alcohol | 3.6 | 3.6 |
| Sodium benzoate | 0.59 | 0.59 |
| Citric acid 1H20 | 0.6 | 0.6 |
| Quartamin BTC-131 | 2.5 | 2.5 |
| Keratin hydrolysate | 0.04 | 0.04 |
| Glycerin (99.5%) | 1.0 | 1.0 |
| D-Panthenol (75%) | 0.2 | 0.2 |
| Apricot kernel oil | 0.4 | 0.4 |
| Isopropyl myristate | 0.8 | 0.8 |
| Fragrance | 0.35 | 0.35 |
| Water | ad 100 | ad 100 |

The trained hairdressers arrived at the following ratings:

| | Hair conditioner according |
|---|---|
| Test parameter | to the invention |
| Feel of the hair during rinse-out | 0.8 |
| Ease of untangling the hair (wet) | 0.3 |
| Wet combability | 0.6 |
| Workability of the hair with the blow | 0.1 |
| dryer | |
| Ease of untangling dry (only with the | 0.2 |
| dryer hood) | |
| Dry combability | 0.2 |

The rating values were based on a scale of zero to 1. Values less than 0.25 represent statistically insignificant differences. Values from 0.25 to 0.5 represent a slight improvement over the comparative formulation. Values greater than 0.5 represent a statistically significant improvement over the comparative formulation.

The formulation according to the invention aided the treated hair with regard to improved feel and luster of the dry hair. This effect was ascertainable even after a further hair washing and drying, without application of conditioner (test parameter: “long-term effect”).

Test Parameter

| | |
|---|---|
| Ease of rinsing out | 0.2 |
| Ease of untangling hair (wet) | 0.1 |
| Wet combability | 0.1 |
| Workability of the hair with the blow dryer | 0.1 |
| Feel (dry) | 0.4 |
| Feel of the hair ends (dry) | 0.4 |
| Long-term effect | 0.5 |
| Luster | 0.4 |
| Overall rating | 0.5 |

Raw Materials Used

| | |
|---|---|
| Dehyquart F75 | Distearoylethyl hydroxyethylmonium methosulfate |
| | (68.5% by weight), |
| | cetearyl alcohol (31.5% by weight) |
| Cutina GMS V | Glycerol mono/dipalmitate/stearate; approximately |
| | 1:1 mixture of glycerin mono- and diesters |
| Quartamin BTC-131 | Behenoyl PG-trimonium chloride (70% by |
| | weight), hexylene glycol (2-methylpentanediol-2,4, |
| | 17% by weight), water (12.2% by weight), citric |
| | acid (0.8% by weight) |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic composition for conditioning keratin fibers, including in a cosmetic carrier
a) at least one cationic surfactant of formula (Tkat1-3), 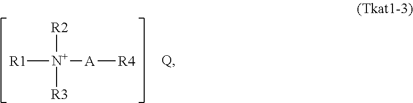
wherein R1, R2, and R3 each independently are:
hydrogen, with the condition that R4 is R8-NH—, or
a free electron pair, with the condition that R4 is R8-NH—, and only one of the moieties R1, R2, R3 is a free electron pair and Q is not required, or
a branched or linear alkyl moiety, including 1 to 4 carbon atoms, which may include at least one hydroxyl group, or
a saturated or unsaturated, branched or linear alkyl moiety including 6 to 30 carbon atoms, which may include at least one hydroxyl group, or
the moiety (-A-R4), with the condition that no more than two of the moieties R1, R2, or R3 may be this moiety, or
R2 and R3 form a cyclic or heterocyclic, optionally unsaturated alkylene group which may be substituted with a saturated or unsaturated, branched or linear alkyl moiety including 6 to 20 carbon atoms,
and A stands for:
1) —(CH₂)ₙ—, wherein n=1 to 20, or
2) —(CH₂—CHR₅—O)ₙ, wherein n=1 to 200, and wherein R5 is hydrogen, methyl, or ethyl,
3) a hydroxyalkylene group including one to four carbon atoms, which may be branched or linear and which include one to three hydroxy groups,
and R4 is:
R6-COO—, wherein R6 is a saturated or unsaturated, branched or linear alkyl moiety or a cyclic, saturated or unsaturated alkyl moiety including 6 to 30 carbon atoms, which includes at least one hydroxy group and optionally may be oxalkylated with 1 to 100 ethylene oxide and/or propylene oxide units, or
R7-CO—, wherein R7 is a saturated or unsaturated, branched or linear alkyl moiety or a cyclic, saturated or unsaturated alkyl moiety including 6 to 30 carbon atoms, which may include at least one hydroxy group and which may optionally also be oxalkylated with 1 to 100 ethylene oxide and/or propylene oxide units, or
R8-NH—, where R8 is a linear or branched, acyl or alkyl moiety in each case including 6 to 30 C atoms, which may be saturated or unsaturated and/or substituted with at least one OH group, and
Q is a physiologically compatible organic or inorganic anion, if none of the moieties R1, R2, R3 stands for a free electron pair,
and in addition
b) at least one ester of 3-methyl-1-butanol with an acid of the general formula R′COOH, wherein R′ represents a saturated or unsaturated, branched or linear alkyl moiety including 7 to 29 carbon atoms, and which may include at least one hydroxy group,
wherein the at least one cationic surfactant of formula (Tkat1-3) is included in a total quantity of 0.2 to 10.0% by weight based on the weight of the composition.

2. The cosmetic composition according to claim 1, wherein
R1 and R2 and R3 are methyl and
A is a linear hydroxyalkylene group which includes three carbon atoms and has a hydroxy group, and
R4 is R6-COO—,
R6 stands for a saturated, linear alkyl moiety including 11 to 21 carbon atoms, and
Q stands for methosulfates, ethosulfates, or chloride.

3. The cosmetic composition according to claim 1, wherein the at least one cationic surfactant is 3-behenoyloxy-2-hydroxypro-pyltrimethylammonium chloride.

4. The cosmetic composition according to claim 1, wherein
R1 is methyl,
R2 is 2-hydroxyethyl,
R3 is the moiety (-A-R4),
A is —CH₂—CH₂—,
R4 is R6-COO—,
R6 is a saturated, linear alkyl moiety including 11 to 21 carbon atoms, and
Q is a methosulfate, ethosulfate, or chloride.

5. The cosmetic composition according to claim 1, wherein the at least one cationic surfactant is distearoylethyl hydroxyethylmonium methosulfate.

6. The cosmetic composition according to claim 1, wherein
R1 stands for methyl,
R2 and R3 form a heterocyclic unsaturated alkylene group which is substituted with a saturated or unsaturated, linear alkyl moiety including 6 to 20 carbon atoms,
A is —CH₂—CH₂—,
R4 is R8-NH—,
R8 is a linear acyl moiety including 6 to 30 C atoms, which is saturated or unsaturated, and
Q is a methosulfate, ethosulfate, or chloride.

7. The cosmetic composition according to claim 1, wherein the at least one cationic surfactant is selected from the group consisting of Quaternium-87 and Quaternium-27.

8. The cosmetic composition according to claim 1, wherein
R1 and R2 are methyl,
R3 is hydrogen or a free electron pair,
A is —CH₂—CH₂—CH₂—,
R4 is R8-NH—,
R8 is a linear or branched acyl moiety including 6 to 30 C atoms, which is saturated or unsaturated, and
Q is a methosulfate or ethosulfate, or a chloride if R3 is hydrogen.

9. The cosmetic composition according to claim 1, wherein the at least one cationic surfactant is at least one amidoamine or at least one permanent cationic amidoamine.

10. The cosmetic composition according to claim 9, wherein the at least one cationic surfactant is selected from the group consisting of lauramidopropyl dimethylamine, myristamidopropyl dimethylamine, stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine, palmamidopropyl dimethylamine, Quaternium-33, behenamidopropyl ethyldimonium ethosulfate, oleamidopropyl ethyldimonium ethosulfate, cocamidopropyltrimonium chloride, rinoleamidopropyltrimonium chloride, stearamidopropyl trimonium methosulfate, and mixtures thereof, preferably selected from amidoamine stearamidopropyl dimethylamine, cocamidopropyl dimethylamine, ricinolamidopropyl dimethylamine, isostearamidopropyl dimethylamine, oleamidopropyl dimethylamine, behenamidopropyl dimethylamine, palmamidopropyl dimethylamine, Quaternium-33, and behenamidopropyl ethyldimonium ethosulfate.

11. The cosmetic composition according to claim 1, wherein the at least one ester of 3-methyl-1-butanol with an acid of the general formula R′COOH is isoamyl laurate and is included in a total quantity of 0.1 to 5% by weight of the composition.

12. A method for hair treatment, including applying the cosmetic agent according to claim 1 to the hair, and rinsing the agent from the hair after an exposure period of 5 seconds to 100 minutes.

13. A method for hair treatment, including applying the cosmetic agent according to claim 1 to the hair for more than 100 minutes and until the next hair washing.
